SEQUENCE LISTING (4 sequences)

SEQ ID NO: 1 - protein, 21 aa, Artificial Sequence
  source: /note=“Description of Artificial Sequence Synthetic peptide”
NAVPNLRGDLQVLAQKVARTC

SEQ ID NO: 2 - protein, 21 aa, Artificial Sequence
  source: /note=“Description of Artificial Sequence Synthetic peptide”
  MOD_RES (4)..(18): Any amino acid
NAVXXXXXXXXXXXXXXXRTC

SEQ ID NO: 3 - protein, 4 aa, Artificial Sequence
  source: /note=“Description of Artificial Sequence Synthetic peptide”
GGFG

SEQ ID NO: 4 - protein, 4 aa, Artificial Sequence
  source: /note=“Description of Artificial Sequence Synthetic peptide”
GFGG

(19) United States
(12) Patent Application Publication
(10) Pub. No.: US 2023/0097908 A1
(43) Pub. Date: Mar. 30, 2023

(54) COMPOUNDS AND CONJUGATES THEREOF

(71) Applicant: MEDIMMUNE LIMITED, Cambridge (GB)

(72) Inventors: Philip Wilson HOWARD, Cambridge (GB); Thais CAILLEAU, Cambridge (GB)

(21) Appl. No.: 17/759,083

(22) PCT Filed: Jan. 21, 2021
(86) PCT No.: PCT/EP2021/051259

§ 371 Date: Jul. 19, 2022

Related U.S. Application Data
(60) Provisional application No. 62/964,181, filed on Jan. 22, 2020.

(51) Int. Cl. A61K 47/68; A61K 47/55; A61K 47/54

(52) U.S. Cl. CPC A61K 47/6803; A61K 47/55; A61K 47/545; A61K 47/6855; A61K 47/6889

(57) ABSTRACT

A conjugate comprising the following topoisomerase inhibitor derivative (A*): where Y is H or F, with a single overall linker moiety connecting two topoisomerase inhibitor derivatives to a Ligand Unit, wherein the topoisomerase inhibitor derivatives are cleavable from the Ligand Unit. Also provided is A* with the linking unit attached, and intermediates for their synthesis.

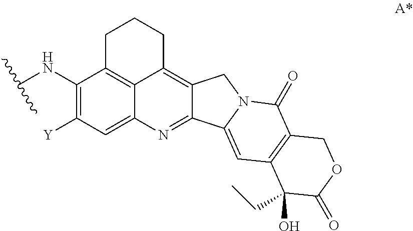

The present invention relates to targeted conjugates comprising topoisomerase inhibitors and compounds useful in their synthesis.

### BACKGROUND TO THE INVENTION

Topoisomerase Inhibitors

Topoisomerase inhibitors are chemical compounds that block the action of topoisomerase (topoisomerase I and II), which is a type of enzyme that controls the changes in DNA structure by catalyzing the breaking and rejoining of the phosphodiester backbone of DNA strands during the normal cell cycle.

The Following Compound:

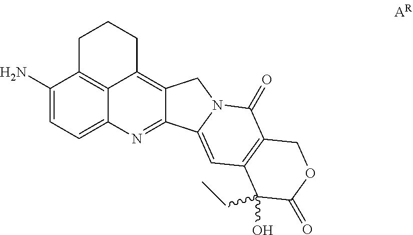

in racemic form was disclosed in EP 0296597 (Example 63). It is also disclosed (as compound 34 in racemic form) in Sugimori, M., et al., J Med Chem, 1998, 41, 2308-2318 (DOI: 10.1021/jm970765q), where its biological activity is discussed, alongside that of a number of related compounds.

Various topoisomerase inhibitors, such as irinotecan and exatecan derivatives and doxorubicin, have been included in antibody drug conjugates. For example, Daiichi Sankyo have DS-8201a in clinical trials:

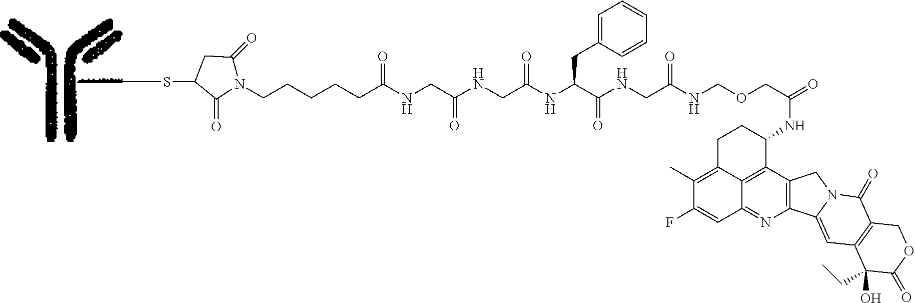 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)[C@H](Cc1ccccc1)NC(=O)CNC(=O)CNC(=O)CCCCCN1C(=O)CC(SC)C1=O)CC3]

where the antibody is Her2 (Takegawa, N., et al., Int J Cancer, 2017, 141, 1682-1689 (DOI: 10.1002/ijc.30870). This ADC releases the exatecan derivative:

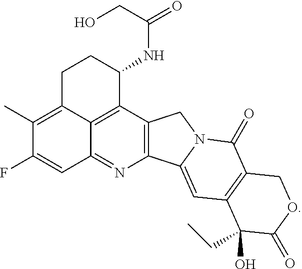 [SMILES: CC[C@@]1(O)C(=O)OCc2c1cc1n(c2=O)Cc2c-1nc1cc(F)c(C)c3c1c2[C@@H](NC(=O)CO)CC3]

Burke, P. J., et al., Bioconjugate Chem., 2009, 20, 1242-1250, discloses conjugates of:

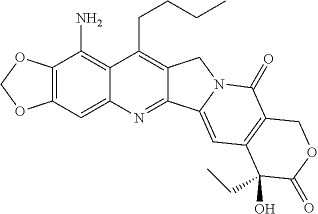 [SMILES: CCCCc1c2c(nc3cc4c(c(N)c13)OCO4)-c1cc3c(c(=O)n1C2)COC(=O)[C@]3(O)CC]

which are linked via the amino group with the following structures:

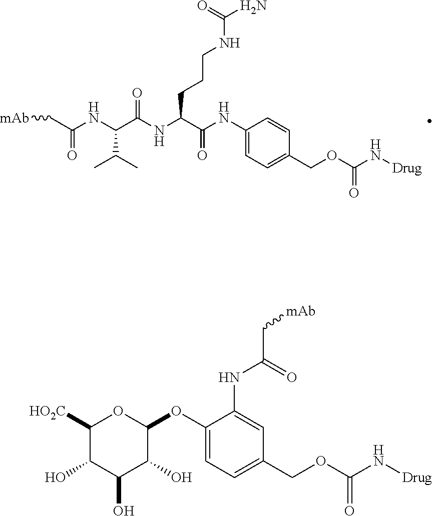

which include a PABC (para-aminobenzyloxycarbonyl) group.

Immunomedics have Sacituzumab Govitecan (IMMU-132) in clinical trials (Cardillo, T. M., et al., Bioconjugate Chem, 2015, 26(5), 919-931, DOI: 10.1021/acs.bioconjchem.5b00223)

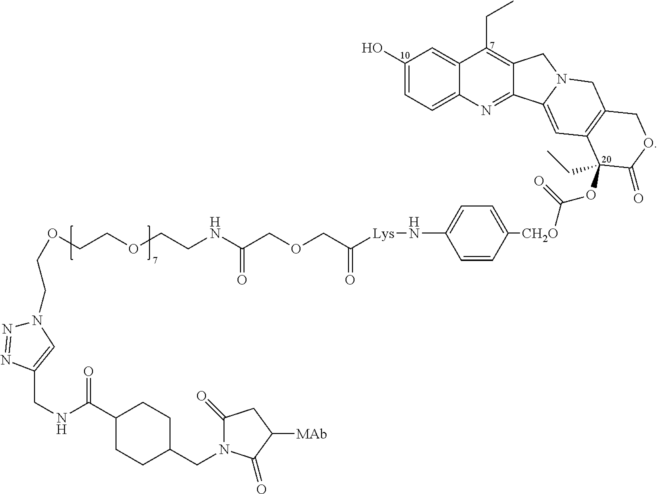

### SUMMARY OF THE INVENTION

In a general aspect the present invention provides a conjugate comprising the following topoisomerase inhibitor derivative (A*, a Drug Unit):

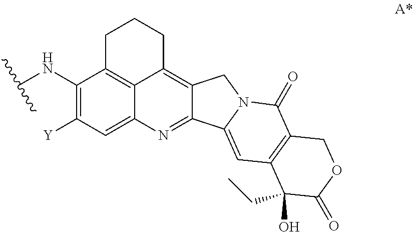

where Y is H or F, with a single overall linker moiety connecting two topoisomerase inhibitor derivatives to a Ligand Unit, wherein the topoisomerase inhibitor derivatives are cleavable from the Ligand Unit. The Ligand Unit is preferably an antibody. The invention also provides A* with the linking unit attached, and intermediates for their synthesis.

A first aspect of the present invention comprises a compound with the formula I:

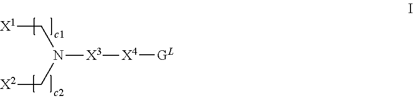

where X¹ and X² are independently selected from a group of formula Ia:

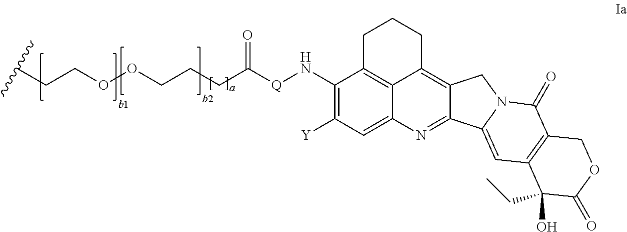

Q is:

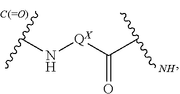

where Q^{X} is such that Q is an amino-acid residue, a dipeptide residue, a tripeptide residue or a tetrapeptide residue;

a=0 to 5, b1=0 to 16 and b2=0 to 16, wherein at least b1 or b2=0 (i.e. only one of b1 and b2 may not be 0);

Y is H or F;

c1 is 0 to 5;

c2 is 0 to 5;

X³ is —CH₂— or —C(═O)—;

X⁴ is ^{X3}—(CH₂)_{d1}—(C₂H₄O)ₑ—(CH₂)_{d2}—^{GL}, where d1 is 0 to 5, d2 is 0 to 5 and e is 0 to 16;

and

G^{L} is a linker for connecting to a Ligand Unit.

A second aspect of the present invention provides a method of making a compound of the first aspect of the invention, comprising at least one of the method steps set out below.

In a third aspect, the present invention provides a conjugates of formula IV:

L−(D^{L})_{P} (IV)

or a pharmaceutically acceptable salt or solvate thereof, wherein L is a Ligand unit (i.e., a targeting agent), D^{L} is a Drug Linker unit that is of formula III:

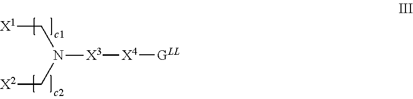

where X¹ and X² are independently selected from a group of formula Ia as defined in the first aspect; X³, X⁴, c1 and c2 are as defined in the first aspect;

G^{LL} is a linker connected to a Ligand Unit; and

p is an integer of from 1 to 20.

Accordingly, the Conjugates comprise a Ligand unit covalently linked to a pair of Drug units (A*) by a Linker unit (i.e. a Ligand unit with one or more Drug-Linker units attached). The Ligand unit, described more fully below, is a targeting agent that binds to a target moiety. The Ligand unit can, for example, specifically bind to a cell component (a Cell Binding Agent) or to other target molecules of interest. Accordingly, the present invention also provides methods for the treatment of, for example, various cancers and autoimmune disease. These methods encompass the use of the Conjugates wherein the Ligand unit is a targeting agent that specifically binds to a target molecule. The Ligand unit can be, for example, a protein, polypeptide or peptide, such as an antibody, an antigen-binding fragment of an antibody, or other binding agent, such as an Fc fusion protein.

The drug loading is represented by twice the value of p, the number of drug units per Ligand unit (e.g., an antibody). Drug loading may range from 2 to 40 Drug units (D) per Ligand unit (e.g., Ab or mAb). For compositions, p represents half the average drug loading of the Conjugates in the composition, and p ranges from 1 to 20.

A fourth aspect of the present invention provides the use of a conjugate of the third aspect of the invention in the manufacture of a medicament for treating a proliferative disease. The fourth aspect also provides a conjugate of the third aspect of the invention for use in the treatment of a proliferative disease.

One of ordinary skill in the art is readily able to determine whether or not a candidate compound treats a proliferative condition for any particular cell type. For example, assays which may conveniently be used to assess the activity offered by a particular compound are described in the examples below.

In Nakada, et al., Bioorg Med Chem Lett, 26 (2016), 1542-1545 (DOI: 10.1016/j.bmc1.2016.02.020) discusses a series of ADCs:

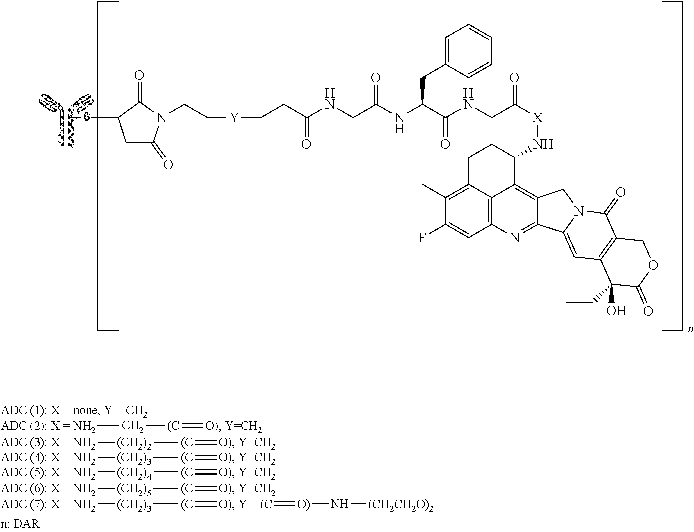

and concludes that the descreased cytotoxicity of ADCs (1) and (2) may be due to the steric hinderance of the released drug moiety on the site acted on by the degrading enzymes in tumour cells. This document teaches the importance of spacing the peptidic group from the bulky released drug moiety. In contrast, in the present invention, the peptidic group is linked directly to the bulky released drug moiety.

Furthermore, the use of a branched linker allows for attachment of more drug units per antibody than with a direct linker. This may be especially useful for use with an engineered antibody, with a limited number of conjugation sides. For example, the branched linker can be used to achieve DAR=4 where the antibody has two engineered cysteines. For antibodies without engineering, with, for example, 8 available cysteines, DAR=16 could be achieved.

### Definitions

C₅₋₆ arylene: The term “C₅₋₆ arylene”, as used herein, pertains to a divalent moiety obtained by removing two hydrogen atoms from an aromatic ring atom of an aromatic compound.

In this context, the prefixes (e.g. C₅₋₆) denote the number of ring atoms, or range of number of ring atoms, whether carbon atoms or heteroatoms.

The ring atoms may be all carbon atoms, as in “carboarylene groups”, in which case the group is phenylene (C₆).

Alternatively, the ring atoms may include one or more heteroatoms, as in “heteroarylene groups”. Examples of heteroarylene groups include, but are not limited to, those derived from:

N₁: pyrrole (azole) (C₅), pyridine (azine) (C₆);

O₁: furan (oxole) (C₅);

S₁: thiophene (thiole) (C₅);

N₁O₁: oxazole (C₅), isoxazole (C₅), isoxazine (C₆);

N₂O₁: oxadiazole (furazan) (C₅);

N₃O₁: oxatriazole (C₅);

N₁S₁: thiazole (C₅), isothiazole (C₅);

N₂: imidazole (1,3-diazole) (C₅), pyrazole (1,2-diazole) (C₅), pyridazine (1,2-diazine) (C₆), pyrimidine (1,3-diazine) (C₆) (e.g., cytosine, thymine, uracil), pyrazine (1,4-diazine) (C₆); and

N₃: triazole (C₅), triazine (C₆).

C₁₋₄ alkyl: The term “C₁₋₄ alkyl” as used herein, pertains to a monovalent moiety obtained by removing a hydrogen atom from a carbon atom of a hydrocarbon compound having from 1 to 4 carbon atoms, which may be aliphatic or alicyclic, and which may be saturated or unsaturated (e.g. partially unsaturated, fully unsaturated). The term “C₁₋ₙ alkyl” as used herein, pertains to a monovalent moiety obtained by removing a hydrogen atom from a carbon atom of a hydrocarbon compound having from 1 to n carbon atoms, which may be aliphatic or alicyclic, and which may be saturated or unsaturated (e.g. partially unsaturated, fully unsaturated). Thus, the term “alkyl” includes the sub-classes alkenyl, alkynyl, cycloalkyl, etc., discussed below.

Examples of saturated alkyl groups include, but are not limited to, methyl (C₁), ethyl (C₂), propyl (C₃) and butyl (C₄).

Examples of saturated linear alkyl groups include, but are not limited to, methyl (C₁), ethyl (C₂), n-propyl (C₃) and n-butyl (C₄).

Examples of saturated branched alkyl groups include iso-propyl (C₃), iso-butyl (C₄), sec-butyl (C₄) and tert-butyl (C₄).

C₂₋₄ Alkenyl: The term “C₂₋₄ alkenyl” as used herein, pertains to an alkyl group having one or more carbon-carbon double bonds.

Examples of unsaturated alkenyl groups include, but are not limited to, ethenyl (vinyl, —CH═CH₂), 1-propenyl (—CH═CH—CH₃), 2-propenyl (allyl, —CH—CH═CH₂), isopropenyl (1-methylvinyl, —C(CH₃)=CH₂) and butenyl (C₄).

C₂₋₄ alkynyl: The term “C₂₋₄ alkynyl” as used herein, pertains to an alkyl group having one or more carbon-carbon triple bonds.

Examples of unsaturated alkynyl groups include, but are not limited to, ethynyl (—C≡CH) and 2-propynyl (propargyl, —CH₂—C≡CH).

C₃₋₄ cycloalkyl: The term “C₃₋₄ cycloalkyl” as used herein, pertains to an alkyl group which is also a cyclyl group; that is, a monovalent moiety obtained by removing a hydrogen atom from an alicyclic ring atom of a cyclic hydrocarbon (carbocyclic) compound, which moiety has from 3 to 7 carbon atoms, including from 3 to 7 ring atoms.

Examples of cycloalkyl groups include, but are not limited to, those derived from:
saturated monocyclic hydrocarbon compounds:

cyclopropane (C₃) and cyclobutane (C₄); and
unsaturated monocyclic hydrocarbon compounds:

cyclopropene (C₃) and cyclobutene (C₄).

Connection labels: In the formula

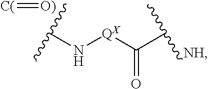

the superscripted labels ^{C(═O)} and ^{NH} indicate the group to which the atoms are bound. For example, the NH group is shown as being bound to a carbonyl (which is not part of the moiety illustrated), and the carbonyl is shown as being bound to a NH group (which is not part of the moiety illustrated).

Salts

It may be convenient or desirable to prepare, purify, and/or handle a corresponding salt of the active compound, for example, a pharmaceutically-acceptable salt. Examples of pharmaceutically acceptable salts are discussed in Berge, et al., J. Pharm. Sci., 66, 1-19 (1977).

For example, if the compound is anionic, or has a functional group which may be anionic (e.g. —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al⁺³. Examples of suitable organic cations include, but are not limited to, ammonium ion (i.e. NH₄⁺) and substituted ammonium ions (e.g. NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

If the compound is cationic, or has a functional group which may be cationic (e.g. —NH₂ may be —NH₃⁺), then a salt may be formed with a suitable anion. Examples of suitable inorganic anions include, but are not limited to, those derived from the following inorganic acids: hydrochloric, hydrobromic, hydroiodic, sulfuric, sulfurous, nitric, nitrous, phosphoric, and phosphorous.

Examples of suitable organic anions include, but are not limited to, those derived from the following organic acids: 2-acetyoxybenzoic, acetic, ascorbic, aspartic, benzoic, camphorsulfonic, cinnamic, citric, edetic, ethanedisulfonic, ethanesulfonic, fumaric, glucheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, toluenesulfonic, trifluoroacetic acid and valeric. Examples of suitable polymeric organic anions include, but are not limited to, those derived from the following polymeric acids: tannic acid, carboxymethyl cellulose.

Solvates

It may be convenient or desirable to prepare, purify, and/or handle a corresponding solvate of the active compound. The term “solvate” is used herein in the conventional sense to refer to a complex of solute (e.g. active compound, salt of active compound) and solvent. If the solvent is water, the solvate may be conveniently referred to as a hydrate, for example, a mono-hydrate, a di-hydrate, a tri-hydrate, etc.

Isomers

Certain compounds of the invention may exist in one or more particular geometric, optical, enantiomeric, diasteriomeric, epimeric, atropic, stereoisomeric, tautomeric, conformational, or anomeric forms, including but not limited to, cis- and trans-forms; E- and Z-forms; c-, t-, and r-forms; endo- and exo-forms; R—, S—, and meso-forms; D- and L-forms; d- and l-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and halfchair-forms; and combinations thereof, hereinafter collectively referred to as “isomers” (or “isomeric forms”).

The term “chiral” refers to molecules which have the property of non-superimposability of the mirror image partner, while the term “achiral” refers to molecules which are superimposable on their mirror image partner.

The term “stereoisomers” refers to compounds which have identical chemical constitution, but differ with regard to the arrangement of the atoms or groups in space.

“Diastereomer” refers to a stereoisomer with two or more centers of chirality and whose molecules are not mirror images of one another. Diastereomers have different physical properties, e.g. melting points, boiling points, spectral properties, and reactivities. Mixtures of diastereomers may separate under high resolution analytical procedures such as electrophoresis and chromatography.

“Enantiomers” refer to two stereoisomers of a compound which are non-superimposable mirror images of one another.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the invention may contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof such as racemic mixtures, form part of the present invention. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and l or (+) and (−) are employed to designate the sign of rotation of plane-polarized light by the compound, with (−) or I meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity.

“Enantiomerically enriched form” refers to a sample of a chiral substance whose enantiomeric ratio is greater than 50:50 but less than 100:0.

Note that, except as discussed below for tautomeric forms, specifically excluded from the term “isomers”, as used herein, are structural (or constitutional) isomers (i.e. isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl. However, a reference to a class of structures may well include structurally isomeric forms falling within that class (e.g. C₁₋₇ alkyl includes n-propyl and iso-propyl; butyl includes n-, iso-, sec-, and tert-butyl; methoxyphenyl includes ortho-, meta-, and para-methoxyphenyl).

The above exclusion does not pertain to tautomeric forms, for example, keto-, enol-, and enolate-forms, as in, for example, the following tautomeric pairs: keto/enol (illustrated below), imine/enamine, amide/imino alcohol, amidine/amidine, nitroso/oxime, thioketone/enethiol, N-nitroso/hyroxyazo, and nitro/aci-nitro.

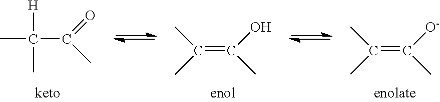

The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons.

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like.

Examples of isotopes that can be incorporated into compounds of the invention include isotopes of hydrogen, carbon, nitrogen, oxygen, phosphorous, fluorine, chlorine and iodine, such as, but not limited to ²H (deuterium, D), ³H (tritium), ¹¹C, ¹³C, ¹⁴C, ¹⁵N, ¹⁸F, ³¹P, ³²P, ³⁵S, ³⁶Cl, and ¹²⁵I. Various isotopically labeled compounds of the present invention, for example those into which radioactive isotopes such as 3H, 13C, and 14C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays, or in radioactive treatment of patients. Deuterium labelled or substituted therapeutic compounds of the invention may have improved DMPK (drug metabolism and pharmacokinetics) properties, relating to distribution, metabolism, and excretion (ADME). Substitution with heavier isotopes such as deuterium may afford certain therapeutic advantages resulting from greater metabolic stability, for example increased in vivo half-life or reduced dosage requirements. An 18F labeled compound may be useful for PET or SPECT studies. Isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent. Further, substitution with heavier isotopes, particularly deuterium (i.e., 2H or D) may afford certain therapeutic advantages resulting from greater metabolic stability, for example increased in vivo half-life or reduced dosage requirements or an improvement in therapeutic index. It is understood that deuterium in this context is regarded as a substituent. The concentration of such a heavier isotope, specifically deuterium, may be defined by an isotopic enrichment factor. In the compounds of this invention any atom not specifically designated as a particular isotope is meant to represent any stable isotope of that atom.

Unless otherwise specified, a reference to a particular compound includes all such isomeric forms, including (wholly or partially) racemic and other mixtures thereof. Methods for the preparation (e.g. asymmetric synthesis) and separation (e.g. fractional crystallisation and chromatographic means) of such isomeric forms are either known in the art or are readily obtained by adapting the methods taught herein, or known methods, in a known manner.

Ligand Unit

The Ligand Unit may be of any kind, and include a protein, polypeptide, peptide and a non-peptidic agent that specifically binds to a target molecule. In some embodiments, the Ligand unit may be a protein, polypeptide or peptide. In some embodiments, the Ligand unit may be a cyclic polypeptide. These Ligand units can include antibodies or a fragment of an antibody that contains at least one target molecule-binding site, lymphokines, hormones, growth factors, or any other cell binding molecule or substance that can specifically bind to a target.

The terms “specifically binds” and “specific binding” refer to the binding of an antibody or other protein, polypeptide or peptide to a predetermined molecule (e.g., an antigen). Typically, the antibody or other molecule binds with an affinity of at least about 1×10⁷ M⁻¹, and binds to the predetermined molecule with an affinity that is at least two-fold greater than its affinity for binding to a non-specific molecule (e.g., BSA, casein) other than the predetermined molecule or a closely-related molecule.

Examples of Ligand units include those agents described for use in WO 2007/085930, which is incorporated herein.

In some embodiments, the Ligand unit is a Cell Binding Agent that binds to an extracellular target on a cell. Such a Cell Binding Agent can be a protein, polypeptide, peptide or a non-peptidic agent. In some embodiments, the Cell Binding Agent may be a protein, polypeptide or peptide. In some embodiments, the Cell Binding Agent may be a cyclic polypeptide. The Cell Binding Agent also may be antibody or an antigen-binding fragment of an antibody. Thus, in one embodiment, the present invention provides an antibody-drug conjugate (ADC).

Cell Binding Agent

A cell binding agent may be of any kind, and include peptides and non-peptides. These can include antibodies or a fragment of an antibody that contains at least one binding site, lymphokines, hormones, hormone mimetics, vitamins, growth factors, nutrient-transport molecules, or any other cell binding molecule or substance.

Peptides

In one embodiment, the cell binding agent is a linear or cyclic peptide comprising 4-30, preferably 6-20, contiguous amino acid residues.

In one embodiment the cell binding agent comprises a peptide that binds integrin αᵥβ₆. The peptide may be selective for αᵥβ₆ over XYS.

In one embodiment the cell binding agent comprises the A20FMDV-Cys polypeptide. The A20FMDV-Cys has the sequence: NAVPNLRGDLQVLAQKVARTC. Alternatively, a variant of the A20FMDV-Cys sequence may be used wherein one, two, three, four, five, six, seven, eight, nine or ten amino acid residues are substituted with another amino acid residue. Furthermore, the polypeptide may have the sequence NAVXXXXXXXXXXXXXXXRTC.

Antibodies

The term “antibody” herein is used in the broadest sense and specifically covers monoclonal antibodies, polyclonal antibodies, dimers, multimers, multispecific antibodies (e.g., bispecific antibodies), multivalent antibodies and antibody fragments, so long as they exhibit the desired biological activity (Miller et al (2003) Jour. of Immunology 170:4854-4861). Antibodies may be murine, human, humanized, chimeric, or derived from other species. An antibody is a protein generated by the immune system that is capable of recognizing and binding to a specific antigen. (Janeway, C., Travers, P., Walport, M., Shlomchik (2001) Immuno Biology, 5th Ed., Garland Publishing, N.Y.). A target antigen generally has numerous binding sites, also called epitopes, recognized by CDRs on multiple antibodies. Each antibody that specifically binds to a different epitope has a different structure. Thus, one antigen may have more than one corresponding antibody. An antibody includes a full-length immunoglobulin molecule or an immunologically active portion of a full-length immunoglobulin molecule, i.e., a molecule that contains an antigen binding site that immunospecifically binds an antigen of a target of interest or part thereof, such targets including but not limited to, cancer cell or cells that produce autoimmune antibodies associated with an autoimmune disease. The immunoglobulin can be of any type (e.g. IgG, IgE, IgM, IgD, and IgA), class (e.g. IgG1, IgG2, IgG3, IgG4, IgA1 and IgA2) or subclass of immunoglobulin molecule. The immunoglobulins can be derived from any species, including human, murine, or rabbit origin.

“Antibody fragments” comprise a portion of a full length antibody, generally the antigen binding or variable region thereof. Examples of antibody fragments include Fab, Fab′, F(ab′)₂, and scFv fragments; diabodies; linear antibodies; fragments produced by a Fab expression library, anti-idiotypic (anti-Id) antibodies, CDR (complementary determining region), and epitope-binding fragments of any of the above which immunospecifically bind to cancer cell antigens, viral antigens or microbial antigens, single-chain antibody molecules; and multispecific antibodies formed from antibody fragments.

The term “monoclonal antibody” as used herein refers to an antibody obtained from a population of substantially homogeneous antibodies, i.e. the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al (1975) Nature 256:495, or may be made by recombinant DNA methods (see, U.S. Pat. No. 4,816,567).

The monoclonal antibodies may also be isolated from phage antibody libraries using the techniques described in Clackson et al (1991) Nature, 352:624-628; Marks et al (1991) J. Mol. Biol., 222:581-597 or from transgenic mice carrying a fully human immunoglobulin system (Lonberg (2008) Curr. Opinion 20(4):450-459).

The monoclonal antibodies herein specifically include chimeric antibodies, humanized antibodies and human antibodies.

Examples of cell binding agents include those agents described for use in WO 2007/085930, which is incorporated herein.

Tumour-associate antigens and cognate antibodies for use in embodiments of the present invention are listed below, and are described in more detail on pages 14 to 86 of WO 2017/186894, which is incorporated herein.

(1) BMPR1B (bone morphogenetic protein receptor-type IB)

(2) E16 (LAT1, SLC7A5)

(3) STEAP1 (six transmembrane epithelial antigen of prostate)

(4) 0772P (CA125, MUC16)

(5) MPF (MPF, MSLN, SMR, megakaryocyte potentiating factor, mesothelin)

(6) Napi3b (NAPI-3B, NPTIIb, SLC34A2, solute carrier family 34 (sodium phosphate), member 2, type II sodium-dependent phosphate transporter 3b)

(7) Sema 5b (FLJ10372, KIAA1445, Mm.42015, SEMA5B, SEMAG, Semaphorin 5b Hlog, sema domain, seven thrombospondin repeats (type 1 and type 1-like), transmembrane domain (TM) and short cytoplasmic domain, (semaphorin) 5B)

(8) PSCA hlg (2700050C12Rik, C530008O16Rik, RIKEN cDNA 2700050C12, RIKEN cDNA 2700050C12 gene)

(9) ETBR (Endothelin type B receptor)

(10) MSG783 (RNF124, hypothetical protein FLJ20315)

(11) STEAP2 (HGNC_8639, IPCA-1, PCANAP1, STAMP1, STEAP2, STMP, prostate cancer associated gene 1, prostate cancer associated protein 1, six transmembrane epithelial antigen of prostate 2, six transmembrane prostate protein)

(12) TrpM4 (BR22450, FLJ20041, TRPM4, TRPM4B, transient receptor potential cation 5 channel, subfamily M, member 4)

(13) CRIPTO (CR, CR1, CRGF, CRIPTO, TDGF1, teratocarcinoma-derived growth factor)

(14) CD21 (CR2 (Complement receptor 2) or C3DR (C3d/Epstein Barr virus receptor) or Hs.73792)

(15) CD79b (CD79B, CD79β, IGb (immunoglobulin-associated beta), B29)

(16) FcRH2 (IFGP4, IRTA4, SPAP1A (SH2 domain containing phosphatase anchor protein 1a), SPAP1B, SPAP1C)

(17) HER2 (ErbB2)

(18) NCA (CEACAM6)

(19) MDP (DPEP1)

(20) IL20R-alpha (IL20Ra, ZCYTOR7)

(21) Brevican (BCAN, BEHAB)

(22) EphB2R (DRT, ERK, HekS, EPHT3, TyroS)

(23) ASLG659 (B7h)

(24) PSCA (Prostate stem cell antigen precursor)

(25) GEDA

(26) BAFF-R (B cell-activating factor receptor, BLyS receptor 3, BR3)

(27) CD22 (B-cell receptor CD22-B isoform, BL-CAM, Lyb-8, Lyb8, SIGLEC-2, FLJ22814) (27a) CD22 (CD22 molecule)

(28) CD79a (CD79A, CD79alpha), immunoglobulin-associated alpha, a B cell-specific protein that covalently interacts with Ig beta (CD79B) and forms a complex on the surface with Ig M molecules, transduces a signal involved in B-cell differentiation), pl: 4.84, MW: 25028 TM: 2 [P] Gene Chromosome: 19q13.2).

(29) CXCR5 (Burkitt's lymphoma receptor 1, a G protein-coupled receptor that is activated by the CXCL13 chemokine, functions in lymphocyte migration and humoral defense, plays a role in HIV-2 infection and perhaps development of AIDS, lymphoma, myeloma, and leukemia); 372 aa, pl: 8.54 MW: 41959 TM: 7 [P] Gene Chromosome: 11q23.3,

(30) HLA-DOB (Beta subunit of MHC class II molecule (la antigen) that binds peptides and 20 presents them to CD4+ T lymphocytes); 273 aa, pl: 6.56, MW: 30820.TM: 1 [P] Gene Chromosome: 6p21.3)

(31) P2X5 (Purinergic receptor P2X ligand-gated ion channel 5, an ion channel gated by extracellular ATP, may be involved in synaptic transmission and neurogenesis, deficiency may contribute to the pathophysiology of idiopathic detrusor instability); 422 aa), pl: 7.63, MW: 47206 TM: 1 [P] Gene Chromosome: 17p13.3).

(32) CD72 (B-cell differentiation antigen CD72, Lyb-2); 359 aa, pl: 8.66, MW: 40225, TM: 1 5 [P] Gene Chromosome: 9p13.3).

(33) LY64 (Lymphocyte antigen 64 (RP105), type I membrane protein of the leucine rich repeat (LRR) family, regulates B-cell activation and apoptosis, loss of function is associated with increased disease activity in patients with systemic lupus erythematosis); 661 aa, pl: 6.20, MW: 74147 TM: 1 [P] Gene Chromosome: 5q12).

(34) FcRH1 (Fc receptor-like protein 1, a putative receptor for the immunoglobulin Fc domain that contains C2 type Ig-like and ITAM domains, may have a role in B-lymphocyte differentiation); 429 aa, pl: 5.28, MW: 46925 TM: 1 [P] Gene Chromosome: 1q21-1q22)

(35) IRTA2 (Immunoglobulin superfamily receptor translocation associated 2, a putative immunoreceptor with possible roles in B cell development and lymphomagenesis; deregulation of the gene by translocation occurs in some B cell malignancies); 977 aa, pl: 6.88, MW: 106468, TM: 1 [P] Gene Chromosome: 1q21)

(36) TENB2 (TMEFF2, tomoregulin, TPEF, HPP1, TR, putative transmembrane 35 proteoglycan, related to the EGF/heregulin family of growth factors and follistatin); 374 aa)

(37) PSMA-FOLH1 (Folate hydrolase (prostate-specific membrane antigen) 1)

(38) SST (Somatostatin Receptor; note that there are5 subtypes)

(38.1) SSTR2 (Somatostatin receptor 2)

(38.2) SSTR5 (Somatostatin receptor 5)

(38.3) SSTR1

(38.4) SSTR3

(38.5) SSTR4

AvB6—Both subunits (39+40)

(39) ITGAV (Integrin, alpha V)

(40) ITGB6 (Integrin, beta 6)

(41) CEACAM5 (Carcinoembryonic antigen-related cell adhesion molecule 5)

(42) MET (met proto-oncogene; hepatocyte growth factor receptor)

(43) MUC1 (Mucin 1, cell surface associated)

(44) CA9 (Carbonic anhydrase IX)

(45) EGFRvIII (Epidermal growth factor receptor (EGFR), transcript variant 3,

(46) CD33 (CD33 molecule)

(47) CD19 (CD19 molecule)

(48) IL2RA (Interleukin 2 receptor, alpha); NCBI Reference Sequence: NM_000417.2);

(49) AXL (AXL receptor tyrosine kinase)

(50) CD30—TNFRSF8 (Tumor necrosis factor receptor superfamily, member 8)

(51) BCMA (B-cell maturation antigen)—TNFRSF17 (Tumor necrosis factor receptor superfamily, member 17)

(52) CT Ags—CTA (Cancer Testis Antigens)

(53) CD174 (Lewis Y)—FUT3 (fucosyltransferase 3 (galactoside 3(4)-L-fucosyltransferase, Lewis blood group)

(54) CLEC14A (C-type lectin domain family 14, member A; Genbank accession no. NM175060)

(55) GRP78—HSPA5 (heat shock 70 kDa protein 5 (glucose-regulated protein, 78 kDa)

(56) CD70 (CD70 molecule) L08096

(57) Stem Cell specific antigens. For example:
5T4 (see entry (63) below)
CD25 (see entry (48) above)
CD32
LGR5/GPR49
Prominin/CD133

(58) ASG-5

(59) ENPP3 (Ectonucleotide pyrophosphatase/phosphodiesterase 3)

(60) PRR4 (Proline rich 4 (lacrimal))

(61) GCC—GUCY2C (guanylate cyclase 2C (heat stable enterotoxin receptor)

(62) Liv-1—SLC39A6 (Solute carrier family 39 (zinc transporter), member 6)

(63) 5T4, Trophoblast glycoprotein, TPBG—TPBG (trophoblast glycoprotein)

(64) CD56—NCMA1 (Neural cell adhesion molecule 1)

(65) CanAg (Tumor associated antigen CA242) (66) FOLR1 (Folate Receptor 1) (67) GPNMB (Glycoprotein (transmembrane) nmb) (68) TIM-1—HAVCR1 (Hepatitis A virus cellular receptor 1) (69) RG-1/Prostate tumor target Mindin—Mindin/RG-1 (70) B7-H4—VTCN1 (V-set domain containing T cell activation inhibitor 1 (71) PTK7 (PTK7 protein tyrosine kinase 7) (72) CD37 (CD37 molecule) (73) CD138—SDC1 (syndecan 1) (74) CD74 (CD74 molecule, major histocompatibility complex, class II invariant chain) (75) Claudins—CLs (Claudins) (76) EGFR (Epidermal growth factor receptor) (77) Her3 (ErbB3)—ERBB3 (v-erb-b2 erythroblastic leukemia viral oncogene homolog 3 (avian)) (78) RON-MST1R (macrophage stimulating 1 receptor (c-met-related tyrosine kinase)) (79) EPHA2 (EPH receptor A2) (80) CD20—MS4A1 (membrane-spanning 4-domains, subfamily A, member 1) (81) Tenascin C—TNC (Tenascin C) (82) FAP (Fibroblast activation protein, alpha) (83) DKK-1 (Dickkopf 1 homolog (Xenopus laevis) (84) CD52 (CD52 molecule) (85) CS1—SLAMF7 (SLAM family member 7) (86) Endoglin—ENG (Endoglin) (87) Annexin A1—ANXA1 (Annexin A1) (88) V-CAM (CD106)—VCAM1 (Vascular cell adhesion molecule 1)

An additional tumour-associate antigen and cognate antibodies of interest are:

(89) ASCT2 (ASC transporter 2, also known as SLC1A5).

ASCT2 antibodies are described in WO 2018/089393, which is incorporated herein by reference

The cell binding agent may be labelled, for example to aid detection or purification of the agent either prior to incorporation as a conjugate, or as part of the conjugate. The label may be a biotin label. In another embodiment, the cell binding agent may be labelled with a radioisotope.

Methods of Treatment

The conjugates of the present invention may be used in a method of therapy. Also provided is a method of treatment, comprising administering to a subject in need of treatment a therapeutically-effective amount of a conjugate of formula IV. The term “therapeutically effective amount” is an amount sufficient to show benefit to a patient. Such benefit may be at least amelioration of at least one symptom. The actual amount administered, and rate and time-course of administration, will depend on the nature and severity of what is being treated. Prescription of treatment, e.g. decisions on dosage, is within the responsibility of general practitioners and other medical doctors.

A conjugate may be administered alone or in combination with other treatments, either simultaneously or sequentially dependent upon the condition to be treated. Examples of treatments and therapies include, but are not limited to, chemotherapy (the administration of active agents, including, e.g. drugs; surgery; and radiation therapy.

Pharmaceutical compositions according to the present invention, and for use in accordance with the present invention, may comprise, in addition to the active ingredient, i.e. a conjugate of formula IV, a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration, which may be oral, or by injection, e.g. cutaneous, subcutaneous, or intravenous.

Pharmaceutical compositions for oral administration may be in tablet, capsule, powder or liquid form. A tablet may comprise a solid carrier or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included. A capsule may comprise a solid carrier such a gelatin.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

The Conjugates can be used to treat proliferative disease and autoimmune disease. The term “proliferative disease” pertains to an unwanted or uncontrolled cellular proliferation of excessive or abnormal cells which is undesired, such as, neoplastic or hyperplastic growth, whether in vitro or in vivo.

Examples of proliferative conditions include, but are not limited to, benign, pre-malignant, and malignant cellular proliferation, including but not limited to, neoplasms and tumours (e.g., histocytoma, glioma, astrocyoma, osteoma), cancers (e.g. lung cancer, small cell lung cancer, gastrointestinal cancer, bowel cancer, colon cancer, breast carcinoma, ovarian carcinoma, prostate cancer, testicular cancer, liver cancer, kidney cancer, bladder cancer, pancreatic cancer, brain cancer, sarcoma, osteosarcoma, Kaposi's sarcoma, melanoma), leukemias, psoriasis, bone diseases, fibroproliferative disorders (e.g. of connective tissues), and atherosclerosis. Other cancers of interest include, but are not limited to, haematological; malignancies such as leukemias and lymphomas, such as non-Hodgkin lymphoma, and subtypes such as DLBCL, marginal zone, mantle zone, and follicular, Hodgkin lymphoma, AML, and other cancers of B or T cell origin. Any type of cell may be treated, including but not limited to, lung, gastrointestinal (including, e.g. bowel, colon), breast (mammary), ovarian, prostate, liver (hepatic), kidney (renal), bladder, pancreas, brain, and skin.

Examples of autoimmune disease include the following: rheumatoid arthritis, autoimmune demyelinative diseases (e.g., multiple sclerosis, allergic encephalomyelitis), psoriatic arthritis, endocrine ophthalmopathy, uveoretinitis, systemic lupus erythematosus, myasthenia gravis, Graves' disease, glomerulonephritis, autoimmune hepatological disorder, inflammatory bowel disease (e.g., Crohn's disease), anaphylaxis, allergic reaction, Sjögren's syndrome, type I diabetes mellitus, primary biliary cirrhosis, Wegener's granulomatosis, fibromyalgia, polymyositis, dermatomyositis, multiple endocrine failure, Schmidt's syndrome, autoimmune uveitis, Addison's disease, adrenalitis, thyroiditis, Hashimoto's thyroiditis, autoimmune thyroid disease, pernicious anemia, gastric atrophy, chronic hepatitis, lupoid hepatitis, atherosclerosis, subacute cutaneous lupus erythematosus, hypoparathyroidism, Dressler's syndrome, autoimmune thrombocytopenia, idiopathic thrombocytopenic purpura, hemolytic anemia, pemphigus vulgaris, pemphigus, dermatitis herpetiformis, alopecia arcata, pemphigoid, scleroderma, progressive systemic sclerosis, CREST syndrome (calcinosis, Raynaud's phenomenon, esophageal dysmotility, sclerodactyly, and telangiectasia), male and female autoimmune infertility, ankylosing spondolytis, ulcerative colitis, mixed connective tissue disease, polyarteritis nedosa, systemic necrotizing vasculitis, atopic dermatitis, atopic rhinitis, Goodpasture's syndrome, Chagas' disease, sarcoidosis, rheumatic fever, asthma, recurrent abortion, anti-phospholipid syndrome, farmer's lung, erythema multiforme, post cardiotomy syndrome, Cushing's syndrome, autoimmune chronic active hepatitis, bird-fancier's lung, toxic epidermal necrolysis, Alport's syndrome, alveolitis, allergic alveolitis, fibrosing alveolitis, interstitial lung disease, erythema nodosum, pyoderma gangrenosum, transfusion reaction, Takayasu's arteritis, polymyalgia rheumatica, temporal arteritis, schistosomiasis, giant cell arteritis, ascariasis, aspergillosis, Sampter's syndrome, eczema, lymphomatoid granulomatosis, Behcet's disease, Caplan's syndrome, Kawasaki's disease, dengue, encephalomyelitis, endocarditis, endomyocardial fibrosis, endophthalmitis, erythema elevatum et diutinum, psoriasis, erythroblastosis fetalis, eosinophilic faciitis, Shulman's syndrome, Felty's syndrome, filariasis, cyclitis, chronic cyclitis, heterochronic cyclitis, Fuch's cyclitis, IgA nephropathy, Henoch-Schonlein purpura, graft versus host disease, transplantation rejection, cardiomyopathy, Eaton-Lambert syndrome, relapsing polychondritis, cryoglobulinemia, Waldenstrom's macroglobulemia, Evan's syndrome, and autoimmune gonadal failure.

In some embodiments, the autoimmune disease is a disorder of B lymphocytes (e.g., systemic lupus erythematosus, Goodpasture's syndrome, rheumatoid arthritis, and type I diabetes), Th1-lymphocytes (e.g., rheumatoid arthritis, multiple sclerosis, psoriasis, Sjögren's syndrome, Hashimoto's thyroiditis, Graves' disease, primary biliary cirrhosis, Wegener's granulomatosis, tuberculosis, or graft versus host disease), or Th2-lymphocytes (e.g., atopic dermatitis, systemic lupus erythematosus, atopic asthma, rhinoconjunctivitis, allergic rhinitis, Omenn's syndrome, systemic sclerosis, or chronic graft versus host disease). Generally, disorders involving dendritic cells involve disorders of Th1-lymphocytes or Th2-lymphocytes. In some embodiments, the autoimmunie disorder is a T cell-mediated immunological disorder.

A “chemotherapeutic agent” is a chemical compound useful in the treatment of cancer, regardless of mechanism of action. Classes of chemotherapeutic agents include, but are not limited to: alkylating agents, antimetabolites, spindle poison plant alkaloids, cytotoxic/antitumor antibiotics, topoisomerase inhibitors, antibodies, photosensitizers, and kinase inhibitors. Chemotherapeutic agents include compounds used in “targeted therapy” and conventional chemotherapy.

Examples of chemotherapeutic agents include: erlotinib (TARCEVA®, Genentech/OSI Pharm.), docetaxel (TAXOTERE®, Sanofi-Aventis), 5-FU (fluorouracil, 5-fluorouracil, CAS No. 51-21-8), gemcitabine (GEMZAR®, Lilly), PD-0325901 (CAS No. 391210-10-9, Pfizer), cisplatin (cis-diamine, dichloroplatinum(II), CAS No. 15663-27-1), carboplatin (CAS No. 41575-94-4), paclitaxel (TAXOL®, Bristol-Myers Squibb Oncology, Princeton, N.J.), trastuzumab (HERCEPTIN®, Genentech), temozolomide (4-methyl-5-oxo-2,3,4,6,8-pentazabicyclo [4.3.0] nona-2,7,9-triene- 9-carboxamide, CAS No. 85622-93-1, TEMODAR®, TEMODAL®, Schering Plough), tamoxifen ((Z)-2-[4-(1,2-diphenylbut-1-enyl)phenoxy]-N,N-dimethylethanamine, NOLVADEX®, ISTUBAL®, VALODEX®), and doxorubicin (ADRIAMYCIN®), Akti-1/2, HPPD, and rapamycin. More examples of chemotherapeutic agents include: oxaliplatin (ELOXATIN®, Sanofi), bortezomib (VELCADE®, Millennium Pharm.), sutent (SUNITINIB®, SU11248, Pfizer), letrozole (FEMARA®, Novartis), imatinib mesylate (GLEEVEC®, Novartis), XL-518 (Mek inhibitor, Exelixis, WO 2007/044515), ARRY-886 (Mek inhibitor, AZD6244, Array BioPharma, Astra Zeneca), SF-1126 (PI3K inhibitor, Semafore Pharmaceuticals), BEZ-235 (PI3K inhibitor, Novartis), XL-147 (PI3K inhibitor, Exelixis), PTK787/ZK 222584 (Novartis), fulvestrant (FASLODEX®, AstraZeneca), leucovorin (folinic acid), rapamycin (sirolimus, RAPAMUNE®, Wyeth), lapatinib (TYKERB®, GSK572016, Glaxo Smith Kline), lonafarnib (SARASAR™, SCH 66336, Schering Plough), sorafenib (NEXAVAR®, BAY43-9006, Bayer Labs), gefitinib (IRESSA®, AstraZeneca), irinotecan (CAMPTOSAR®, CPT-11, Pfizer), tipifarnib (ZARNESTRA™, Johnson & Johnson), ABRAXANE™ (Cremophor-free), albumin-engineered nanoparticle formulations of paclitaxel (American Pharmaceutical Partners, Schaumberg, II), vandetanib (rINN, ZD6474, ZACTIMA®, AstraZeneca), chloranmbucil, AG1478, AG1571 (SU 5271; Sugen), temsirolimus (TORISEL®, Wyeth), pazopanib (GlaxoSmithKline), canfosfamide (TELCYTA®, Telik), thiotepa and cyclosphosphamide (CYTOXAN®, NEOSAR®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analog topotecan); bryostatin; callystatin; CC-1065 (including its adozelesin, carzelesin and bizelesin synthetic analogs); cryptophycins (particularly cryptophycin 1 and cryptophycin 8); dolastatin; duocarmycin (including the synthetic analogs, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g. calicheamicin, calicheamicin gamma1I, calicheamicin omegal1 (Angew Chem. Intl. Ed. Engl. (1994) 33:183-186); dynemicin, dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, morpholino-doxorubicin, cyanomorpholino-doxorubicin, 2-pyrrolino-doxorubicin and deoxydoxorubicin), epirubicin, esorubicin, idarubicin, nemorubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; 6-thioguanine; mercaptopurine; methotrexate; platinum analogs such as cisplatin and carboplatin; vinblastine; etoposide (VP-16); ifosfamide; mitoxantrone; vincristine; vinorelbine (NAVELBINE®); novantrone; teniposide; edatrexate; daunomycin; aminopterin; capecitabine (XELODA®, Roche); ibandronate; CPT-11; topoisomerase inhibitor RFS 2000; difluoromethylornithine (DMFO); retinoids such as retinoic acid; and pharmaceutically acceptable salts, acids and derivatives of any of the above.

Also included in the definition of “chemotherapeutic agent” are: (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide, and goserelin; as well as troxacitabine (a 1,3-dioxolane nucleoside cytosine analog); (iv) protein kinase inhibitors such as MEK inhibitors (WO 2007/044515); (v) lipid kinase inhibitors; (vi) antisense oligonucleotides, particularly those which inhibit expression of genes in signaling pathways implicated in aberrant cell proliferation, for example, PKC-alpha, Raf and H-Ras, such as oblimersen (GENASENSE®, Genta Inc.); (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN® rIL-2; topoisomerase 1 inhibitors such as LURTOTECAN®; ABARELIX® rmRH; (ix) anti-angiogenic agents such as bevacizumab (AVASTIN®, Genentech); and pharmaceutically acceptable salts, acids and derivatives of any of the above.

Also included in the definition of “chemotherapeutic agent” are therapeutic antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMNITARG™, 2C4, Genentech), trastuzumab (HERCEPTIN®, Genentech), tositumomab (Bexxar, Corixia), and the antibody drug conjugate, gemtuzumab ozogamicin (MYLOTARG®, Wyeth).

Humanized monoclonal antibodies with therapeutic potential as chemotherapeutic agents in combination with the conjugates of the invention include: alemtuzumab, apolizumab, aselizumab, atlizumab, bapineuzumab, bevacizumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pertuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, trastuzumab, tucotuzumab celmoleukin, tucusituzumab, umavizumab, urtoxazumab, and visilizumab.

Formulations

While it is possible for the conjugate to be used (e.g., administered) alone, it is often preferable to present it as a composition or formulation.

In one embodiment, the composition is a pharmaceutical composition (e.g., formulation, preparation, medicament) comprising a conjugate, as described herein, and a pharmaceutically acceptable carrier, diluent, or excipient.

In one embodiment, the composition is a pharmaceutical composition comprising at least one conjugate, as described herein, together with one or more other pharmaceutically acceptable ingredients well known to those skilled in the art, including, but not limited to, pharmaceutically acceptable carriers, diluents, excipients, adjuvants, fillers, buffers, preservatives, anti-oxidants, lubricants, stabilisers, solubilisers, surfactants (e.g., wetting agents), masking agents, colouring agents, flavouring agents, and sweetening agents.

In one embodiment, the composition further comprises other active agents, for example, other therapeutic or prophylactic agents.

Suitable carriers, diluents, excipients, etc. can be found in standard pharmaceutical texts. See, for example, Handbook of Pharmaceutical Additives, 2nd Edition (eds. M. Ash and I. Ash), 2001 (Synapse Information Resources, Inc., Endicott, N.Y., USA), Remington's Pharmaceutical Sciences, 20th edition, pub. Lippincott, Williams & Wilkins, 2000; and Handbook of Pharmaceutical Excipients, 2nd edition, 1994.

Another aspect of the present invention pertains to methods of making a pharmaceutical composition comprising admixing at least one [¹¹C]-radiolabelled conjugate or conjugate-like compound, as defined herein, together with one or more other pharmaceutically acceptable ingredients well known to those skilled in the art, e.g., carriers, diluents, excipients, etc. If formulated as discrete units (e.g., tablets, etc.), each unit contains a predetermined amount (dosage) of the active compound.

The term “pharmaceutically acceptable,” as used herein, pertains to compounds, ingredients, materials, compositions, dosage forms, etc., which are, within the scope of sound medical judgment, suitable for use in contact with the tissues of the subject in question (e.g., human) without excessive toxicity, irritation, allergic response, or other problem or complication, commensurate with a reasonable benefit/risk ratio. Each carrier, diluent, excipient, etc. must also be “acceptable” in the sense of being compatible with the other ingredients of the formulation.

The formulations may be prepared by any methods well known in the art of pharmacy. Such methods include the step of bringing into association the active compound with a carrier which constitutes one or more accessory ingredients. In general, the formulations are prepared by uniformly and intimately bringing into association the active compound with carriers (e.g., liquid carriers, finely divided solid carrier, etc.), and then shaping the product, if necessary.

The formulation may be prepared to provide for rapid or slow release; immediate, delayed, timed, or sustained release; or a combination thereof.

Formulations suitable for parenteral administration (e.g., by injection), include aqueous or non-aqueous, isotonic, pyrogen-free, sterile liquids (e.g., solutions, suspensions), in which the active ingredient is dissolved, suspended, or otherwise provided (e.g., in a liposome or other microparticulate). Such liquids may additional contain other pharmaceutically acceptable ingredients, such as anti-oxidants, buffers, preservatives, stabilisers, bacteriostats, suspending agents, thickening agents, and solutes which render the formulation isotonic with the blood (or other relevant bodily fluid) of the intended recipient. Examples of excipients include, for example, water, alcohols, polyols, glycerol, vegetable oils, and the like. Examples of suitable isotonic carriers for use in such formulations include Sodium Chloride Injection, Ringer's Solution, or Lactated Ringer's Injection. Typically, the concentration of the active ingredient in the liquid is from about 1 ng/ml to about 10 μg/ml, for example from about 10 ng/ml to about 1 μg/ml. The formulations may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules, and tablets.

Dosage

It will be appreciated by one of skill in the art that appropriate dosages of the Conjugates, and compositions comprising the Conjugates, can vary from patient to patient. Determining the optimal dosage will generally involve the balancing of the level of therapeutic benefit against any risk or deleterious side effects. The selected dosage level will depend on a variety of factors including, but not limited to, the activity of the particular compound, the route of administration, the time of administration, the rate of excretion of the compound, the duration of the treatment, other drugs, compounds, and/or materials used in combination, the severity of the condition, and the species, sex, age, weight, condition, general health, and prior medical history of the patient. The amount of compound and route of administration will ultimately be at the discretion of the physician, veterinarian, or clinician, although generally the dosage will be selected to achieve local concentrations at the site of action which achieve the desired effect without causing substantial harmful or deleterious side-effects.

Administration can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell(s) being treated, and the subject being treated. Single or multiple administrations can be carried out with the dose level and pattern being selected by the treating physician, veterinarian, or clinician.

In general, a suitable dose of the active compound is in the range of about 100 ng to about 25 mg (more typically about 1 μg to about 10 mg) per kilogram body weight of the subject per day. Where the active compound is a salt, an ester, an amide, a prodrug, or the like, the amount administered is calculated on the basis of the parent compound and so the actual weight to be used is increased proportionately.

The dosage amounts described above may apply to the conjugate or to the effective amount of compound that is releasable after cleavage of the linker.

For the prevention or treatment of disease, the appropriate dosage of an ADC of the invention will depend on the type of disease to be treated, as defined above, the severity and course of the disease, whether the molecule is administered for preventive or therapeutic purposes, previous therapy, the patient's clinical history and response to the antibody, and the discretion of the attending physician. The molecule is suitably administered to the patient at one time or over a series of treatments. Depending on the type and severity of the disease, about 1 μg/kg to 100 mg/kg or more of molecule is an initial candidate dosage for administration to the patient, whether, for example, by one or more separate administrations, or by continuous infusion. For repeated administrations over several days or longer, depending on the condition, the treatment is sustained until a desired suppression of disease symptoms occurs. Other dosage regimens may be useful. The progress of this therapy is easily monitored by conventional techniques and assays.

Drug Loading

The drug loading is the average number of drugs per Ligand unit, which may be a cell binding agent, e.g. antibody.

The average number of drugs per antibody in preparations of ADC from conjugation reactions may be characterized by conventional means such as UV, reverse phase HPLC, HIC, mass spectroscopy, ELISA assay, and electrophoresis. The quantitative distribution of ADC in terms of p may also be determined. By ELISA, the averaged value of p in a particular preparation of ADC may be determined (Hamblett et al (2004) Clin. Cancer Res. 10:7063-7070; Sanderson et al (2005) Clin. Cancer Res. 11:843-852). However, the distribution of p (drug) values is not discernible by the antibody-antigen binding and detection limitation of ELISA. Also, ELISA assay for detection of antibody-drug conjugates does not determine where the drug moieties are attached to the antibody, such as the heavy chain or light chain fragments, or the particular amino acid residues. In some instances, separation, purification, and characterization of homogeneous ADC where p is a certain value from ADC with other drug loadings may be achieved by means such as reverse phase HPLC or electrophoresis. Such techniques are also applicable to other types of conjugates.

For some antibody-drug conjugates, p may be limited by the number of attachment sites on the antibody. For example, an antibody may have only one or several cysteine thiol groups, or may have only one or several sufficiently reactive thiol groups through which a linker may be attached. Higher drug loading may cause aggregation, insolubility, toxicity, or loss of cellular permeability of certain antibody-drug conjugates.

Typically, fewer than the theoretical maximum of drug moieties are conjugated to an antibody during a conjugation reaction. An antibody may contain, for example, many lysine residues that do not react with the Drug Linker. Only the most reactive lysine groups may react with an amine-reactive linker reagent. Also, only the most reactive cysteine thiol groups may react with a thiol-reactive linker reagent. Generally, antibodies do not contain many, if any, free and reactive cysteine thiol groups which may be linked to a drug moiety. Most cysteine thiol residues in the antibodies of the compounds exist as disulfide bridges and must be reduced with a reducing agent such as dithiothreitol (DTT) or TCEP, under partial or total reducing conditions. The loading (drug/antibody ratio) of an ADC may be controlled in several different manners, including: (i) limiting the molar excess of Drug Linker relative to antibody, (ii) limiting the conjugation reaction time or temperature, and (iii) partial or limiting reductive conditions for cysteine thiol modification.

Certain antibodies have reducible interchain disulfides, i.e. cysteine bridges. Antibodies may be made reactive for conjugation with linker reagents by treatment with a reducing agent such as DTT (dithiothreitol). Each cysteine bridge will thus form, theoretically, two reactive thiol nucleophiles. Additional nucleophilic groups can be introduced into antibodies through the reaction of lysines with 2-iminothiolane (Traut's reagent) resulting in conversion of an amine into a thiol. Reactive thiol groups may be introduced into the antibody (or fragment thereof) by engineering one, two, three, four, or more cysteine residues (e.g., preparing mutant antibodies comprising one or more non-native cysteine amino acid residues). U.S. Pat. No. 7,521,541 teaches engineering antibodies by introduction of reactive cysteine amino acids.

Cysteine amino acids may be engineered at reactive sites in an antibody and which do not form intrachain or intermolecular disulfide linkages (Junutula, et al., 2008b Nature Biotech., 26(8):925-932; Dornan et al (2009) Blood 114(13):2721-2729; U.S. Pat. Nos. 7,521,541; 7,723,485; WO2009/052249). The engineered cysteine thiols may react with Drug-Linkers of the present invention (i.e. of Formula I) which have thiol-reactive, electrophilic groups such as maleimide or alpha-halo amides to form ADC with cysteine engineered antibodies. The location of the drug unit can thus be designed, controlled, and known. The drug loading can be controlled since the engineered cysteine thiol groups typically react with drug-linker reagents in high yield. Engineering an IgG antibody to introduce a cysteine amino acid by substitution at a single site on the heavy or light chain gives two new cysteines on the symmetrical antibody. A drug loading near 4 can be achieved with near homogeneity of the conjugation product ADC.

Where more than one nucleophilic or electrophilic group of the antibody reacts with a Drug-Linkers, then the resulting product may be a mixture of ADC compounds with a distribution of drug units attached to an antibody, e.g. 2, 4, 6, etc. Liquid chromatography methods such as polymeric reverse phase (PLRP) and hydrophobic interaction (HIC) may separate compounds in the mixture by drug loading value. Preparations of ADC with a single drug loading value (p) may be isolated, however, these single loading value ADCs may still be heterogeneous mixtures because the drug units may be attached, via the linker, at different sites on the antibody.

Thus the antibody-drug conjugate compositions of the invention may include mixtures of antibody-drug conjugates where the antibody has one or more drug moieties and where the drug moieties may be attached to the antibody at various amino acid residues.

In one embodiment, the average number of drug linkers per cell binding agent is in the range 1 to 20. In some embodiments the range is selected from 1 to 10, 2 to 10, 2 to 8, 2 to 6, and 4 to 10.

In some embodiments, there are two drugs per cell binding agent.

General Synthetic Routes

Compounds of formula I where X¹ and X² are the same may be synthesised by reacting a compound of Formula 2:

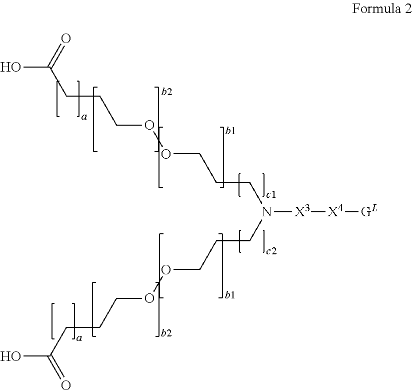

together with two equivalents of a compound of Formula 3:

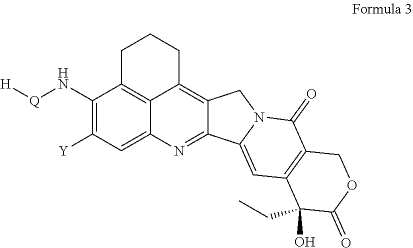

under amide coupling conditions.

Where X¹ and X² are different, each arm of the linker can be constructed separately, using appropriate protection chemistry.

Compounds of formula 3 may be synthesised from a compound of Formula 4:

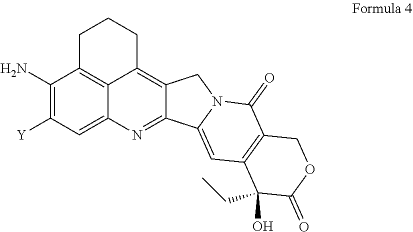

by linking a compound HQH, or a protected version thereof. Such a reaction may be carried out under amide coupling conditions.

Compounds of Formula 4 may be synthesised by the deprotection of a compound of Formula 5:

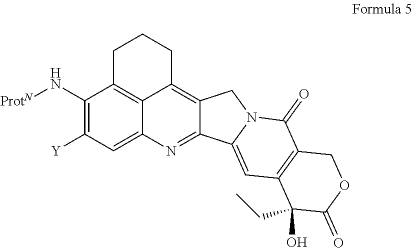

where Prot^{N} is an amine protecting group.

Compounds of Formula 5 may be synthesised by the coupling of a compound of Formula 6:

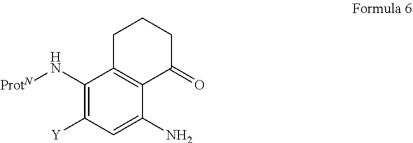

with the compound A3 using the Friedlander reaction.

The synthesis of the compounds of Formula 6 is described in the examples.

Amine Protecting Groups

Amine protecting groups are well-known to those skilled in the art. Particular reference is made to the disclosure of suitable protecting groups in Greene's Protecting Groups in Organic Synthesis, Fourth Edition, John Wiley & Sons, 2007 (ISBN 978-0-471-69754-1), pages 696-871.

Further Preferences

The following preferences may apply to all aspects of the invention as described above, or may relate to a single aspect. The preferences may be combined together in any combination.

Q^{X}

In one embodiment, Q is an amino acid residue. The amino acid may be a natural amino acid or a non-natural amino acid.

In one embodiment, Q is selected from: Phe, Lys, Val, Ala, Cit, Leu, Ile, Arg, and Trp, where Cit is citrulline.

In one embodiment, Q comprises a dipeptide residue. The amino acids in the dipeptide may be any combination of natural amino acids and non-natural amino acids. In some embodiments, the dipeptide comprises natural amino acids. Where the linker is a cathepsin labile linker, the dipeptide is the site of action for cathepsin-mediated cleavage. The dipeptide then is a recognition site for cathepsin.

In one embodiment, Q is selected from:
^{NH}-Phe-Lys-^{C═O},
^{NH}-Val-Ala-^{C═O},
^{NH}-Val-Lys-^{C═O},
^{NH}-Ala-Lys-^{C═O},
^{NH}-Val-Cit-^{C═O},
^{NH}-Phe-Cit-^{C═O},
^{NH}-Leu-Cit-^{C═O},
^{NH}-Ile-Cit-^{C═O},
^{NH}-Phe-Arg-^{C═O},
^{NH}-Trp-Cit-^{C═O}, and
^{NH}-Gly-Val-^{C═O};

where Cit is citrulline. In the above representations of dipeptide residues, NH represents the N-terminus, and -^{C═O} represents the C-terminus of the residue. The C-terminus binds to the NH of A*.

Preferably, Q is selected from:
^{NH}-Phe-Lys-^{C═O},
^{NH}-Val-Ala-^{C═O},
^{NH}-Val-Lys-^{C═O},
^{NH}-Ala-Lys-^{C═O}, and
^{NH}-Val-Cit-^{C═O}.

Most preferably, Q is selected from ^{NH}-Phe-Lys-^{C═O}, ^{NH}-Val-Cit-^{C═O} or ^{NH}Val-Ala-^{C═O}.

Other dipeptide combinations of interest include:
^{NH}-Gly-Gly-^{C═O},
^{NH}-Gly-Val-^{C═O}
^{NH}-Pro-Pro-^{C═O}, and
^{NH}-Val-Glu-^{C═O}.

Other dipeptide combinations may be used, including those described by Dubowchik et al., Bioconjugate Chemistry, 2002, 13,855-869, which is incorporated herein by reference.

In some embodiments, Q is a tripeptide residue. The amino acids in the tripeptide may be any combination of natural amino acids and non-natural amino acids. In some embodiments, the tripeptide comprises natural amino acids. Where the linker is a cathepsin labile linker, the tripeptide is the site of action for cathepsin-mediated cleavage. The tripeptide then is a recognition site for cathepsin. Tripeptide linkers of particular interest are:
^{NH}-Glu-Val-Ala-^{C═O}
^{NH}-Glu-Val-Cit-^{C═O}
^{NH}-αGlu-Val-Ala-^{C═O}
^{NH}-αGlu-Val-Cit-^{C═O}

In some embodiments, Q is a tetrapeptide residue. The amino acids in the tetrapeptide may be any combination of natural amino acids and non-natural amino acids. In some embodiments, the tetrapeptide comprises natural amino acids. Where the linker is a cathepsin labile linker, the tetrapeptide is the site of action for cathepsin-mediated cleavage.

The tetrapeptide then is a recognition site for cathepsin. Tetrapeptide linkers of particular interest are:
^{NH}-Gly-Gly-Phe-Gly ^{C═O}; and
^{NH}-Gly-Gly-Phe-Gly-^{C═O}.

In some embodiments, the tetrapeptide is:
^{NH}-Gly-Gly-Phe-Gly ^{C═O}.

In one embodiment, the amino acid side chain is chemically protected, where appropriate. The side chain protecting group may be a group as discussed above. Protected amino acid sequences are cleavable by enzymes. For example, a dipeptide sequence comprising a Boc side chain-protected Lys residue is cleavable by cathepsin.

Protecting groups for the side chains of amino acids are well known in the art and are described in the Novabiochem Catalog, and as described above.

G^{L}

G^{L} may be selected from

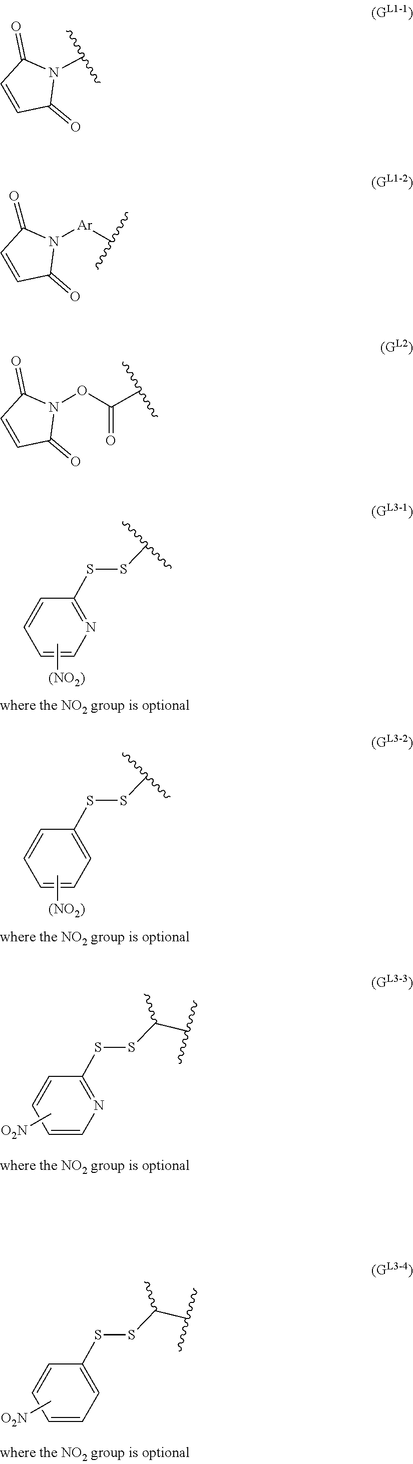

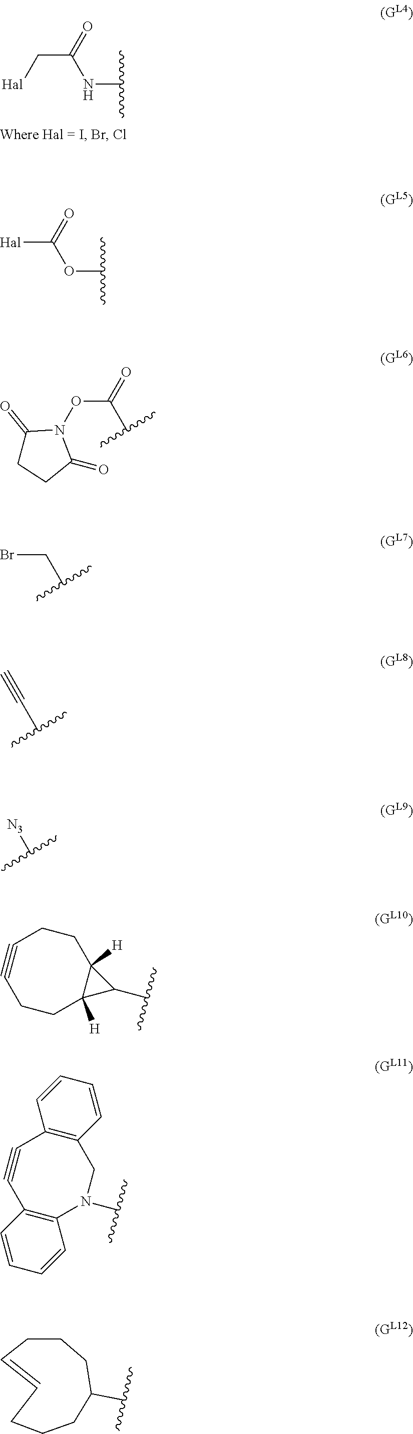

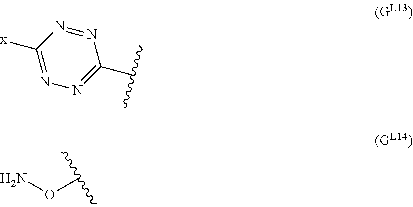

where Ar represents a C₅₋₆ arylene group, e.g. phenylene, and X represents C₁₋₄ alkyl.

In some embodiments, G^{L} is selected from G^{L1-1} and G^{L1-2}. In some of these embodiments, G^{L} is G^{L1-1}.

G^{LL}

G^{LL} may be selected from:

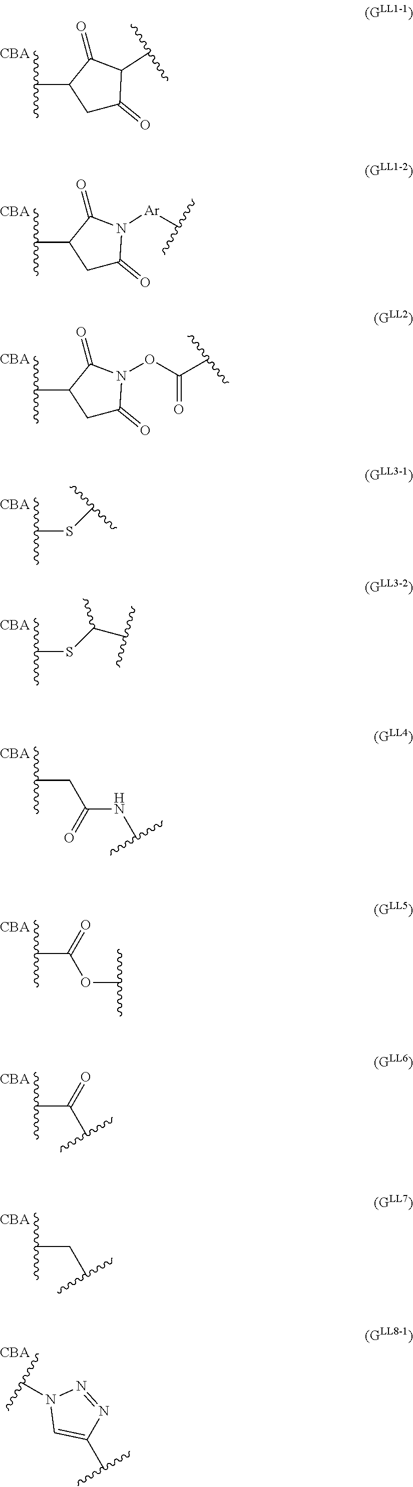

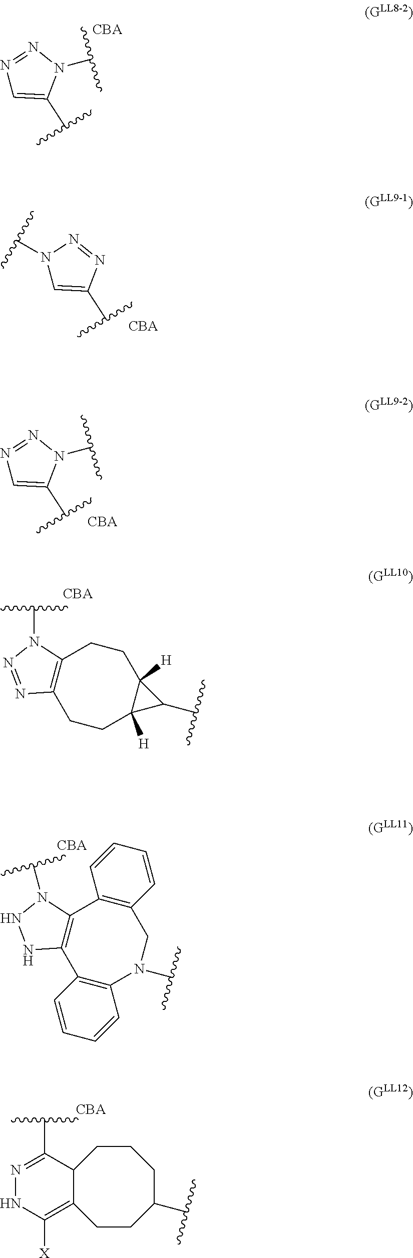

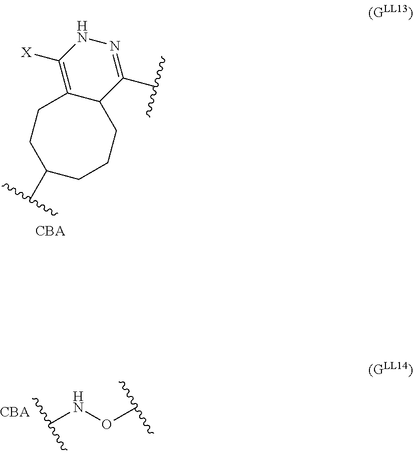

where Ar represents a C₅₋₆ arylene group, e.g. phenylene and X represents C₁₋₄ alkyl.

In some embodiments, G^{LL} is selected from G^{LL1-1} and G^{LL1-2}. In some of these embodiments, G^{LL} is G^{LL1-1}.

In some embodiments, X³ is —CH₂—.

In other embodiments, X³ is —C(═O)—.

In some embodiments, when X³ is —C(═O)—, it is preferred that d1+d2+e≠0.

e may be 0, 1, 2, 3, 4, 5, 6, 7, 8, 9, 10, 11, 12, 13, 14, 15 or 16. In some embodiments, e is 0 to 12. In some of these embodiments, e is 0 to 8, and may be 0, 2, 4 or 8. In some of these embodiments, e is 2. In other of these embodiments, e is 0.

d1 may be 0, 1, 2, 3, 4 or 5. In some embodiments, d1 is 0 to 3. In some of these embodiments, d1 is 1 or 2. In further embodiments, d1 is 2.

d2 may be 0, 1, 2, 3, 4 or 5. In some embodiments, d2 is 0 to 3. In some of these embodiments, d2 is 1 or 2. In further embodiments, d2 is 2. In other embodiments d2 is 0.

In some embodiments d1+d2 is 0 to 5. In some embodiments, d1+d2 is 0 to 3. In some of these embodiments, d1+d2 is 1 or 2. In further embodiments, d1+d2 is 2.

X¹/X²

Each b1 may be 0, 1, 2, 3, 4, 5, 6, 7, 8, 9, 10, 11, 12, 13, 14, 15 or 16. In some embodiments, b1 is 0 to 12. In some of these embodiments, b1 is 0 to 8, and may be 0, 2, 3, 4, 5 or 8. In some of these embodiments, b1 is 2.

Each b2 may be 0, 1, 2, 3, 4, 5, 6, 7, 8, 9, 10, 11, 12, 13, 14, 15 or 16. In some embodiments, b2 is 0 to 12. In some of these embodiments, b2 is 0 to 8, and may be 0, 2, 3, 4, 5 or 8. In some of these embodiments, b2 is 2.

Only one of b1 and b2 may not be 0.

Each a may be 0, 1, 2, 3, 4 or 5. In some embodiments, a is 0 to 3. In some of these embodiments, a is 0 or 1. In further embodiments, a is 0.

In some embodiments of X¹ and X², a is 0 and b is 2.

In some embodiments, Y is H. In other embodiments, Y is F.

In some embodiments, X¹ and X² are the same.

c1 may be 0, 1, 2, 3, 4 or 5. In some embodiments, c1 is 0 to 3. In some of these embodiments, c1 is 1 or 2. In further embodiments, c1 is 2.

c2 may be 0, 1, 2, 3, 4 or 5. In some embodiments, c2 is 0 to 3. In some of these embodiments, c2 is 1 or 2. In further embodiments, c2 is 2.

In some embodiments, c1 and c2 are the same.

In some embodiments, e+the largest value of b1 or b2 may be no more than 16. In some of these embodiments, e+the largest value of b1 or b2 may be no more than 8.

In some embodiments, each a is 0, each b1 is 0, each b2 is 2, c1 is 2, c2=2, X³=—C(═O)—, d1 is 2, d2 is 0 and e is 0. In some of these embodiments, Q is ^{NH}-Val-Ala-^{C═O}.

In some embodiments of the fifth aspect of the invention, the enantiomerically enriched form has an enantiomeric ratio greater than 60:40, 70:30; 80:20 or 90:10. In further embodiments, the enantiomeric ratio is greater than 95:5, 97:3 or 99:1.

### EXAMPLES

Flash chromatography was performed using a Biotage® Isolera™ and fractions checked for purity using thin-layer chromatography (TLC). TLC was performed using Merck Kieselgel 60 F254 silica gel, with fluorescent indicator on aluminium plates. Visualisation of TLC was achieved with UV light or iodine vapour unless otherwise stated.

Extraction and chromatography solvents were bought and used without further purification from VWR U.K or Fisher Scientific, U.K..

All fine chemicals were purchased from Sigma-Aldrich, Lancaster or BDH unless otherwise stated.

LC/MS conditions

Method A

Positive mode electrospray mass spectrometry was performed using a Waters Aquity H-class SQD2.

Mobile phases used were solvent A (water with 0.1% formic acid) and solvent B (acetonitrile with 0.1% formic acid). Initial composition 5% B held over 25 seconds, then increased from 5% B to 100% B over a 1 minute 35 seconds' period. The composition was held for 50 seconds at 100% B, then returned to 5% B in 5 seconds and held there for 5 seconds. The total duration of the gradient run was 3.0 minutes. Flow rate was 0.8 mL/minute. Detection was at 254 nm. Columns: Waters Acquity UPLC® BEH Shield RP18 1.7 μm 2.1×50 mm at 50° C. fitted with Waters Acquity UPLC® BEH Shield RP18 VanGuard Pre-column, 130A, 1.7 μm, 2.1 mm×5 mm.

Method B

The HPLC (Waters Alliance 2695) was run using a mobile phase of water (A) (formic acid 0.1%) and acetonitrile (B) (formic acid 0.1%).

Initial composition 5% B held over 25 seconds, then increased from 5% B to 100% B over a 1 minute 35 seconds' period. The composition was held for 50 seconds at 100% B, then returned to 5% B in 5 seconds and held there for 5 seconds. The total duration of the gradient run was 3.0 minutes. Flow rate was 0.8 mL/minute. Wavelength detection range: 190 to 800 nm. Columns: Waters Acquity UPLC® BEH Shield RP18 1.7 μm 2.1×50 mm at 50° C. fitted with Waters Acquity UPLC® BEH Shield RP18 VanGuard Pre-column, 130A, 1.7 μm, 2.1 mm×5 mm.

Method C

The HPLC (Waters Alliance 2695) was run using a mobile phase of water (A) (formic acid 0.1%) and acetonitrile (B) (formic acid 0.1%).

Initial composition 5% B held over 1 min, then increase from 5% B to 100% B over a 9 min period. The composition was held for 2 min at 100% B, then returned to 5% B in 0.10 minutes and hold there for 3 min. Total gradient run time equals 15 min. Flow rate 0.6 mL/min. Wavelength detection range: 190 to 800 nm. Oven temperature: 50° C. Column: ACE Excel 2 C18-AR, 2 p, 3.0×100 mm.

HPLC Conditions

Reverse-phase ultra-fast high-performance liquid chromatography (UFLC) was carried out on a Shimadzu Prominence™ machine using a Phenomenex™ Gemini NX 5μ C18 column (at 50° C.) dimensions: 150×21.2 mm. Eluents used were solvent A (H₂O with 0.1% formic acid) and solvent B (CH₃CN with 0.1% formic acid). All UFLC experiments were performed with gradient conditions: Initial composition 13% B increased to 30% B over a 3 minutes period, then increased to 45% B over 8 minutes and again to 100% over 6 minutes before retunning to 13% over 2 min and hold for 1 min. The total duration of the gradient run was 20.0 minutes. Flow rate was 20.0 mL/minute and detection was at 254 and 223 nm.

NMR Method

Proton NMR chemical shift values were measured on the delta scale at 400 MHz using a Bruker AV400. The following abbreviations have been used: s, singlet; d, doublet; t, triplet; q, quartet; quin, quintet; m, multiplet; br, broad. Coupling constants are reported in Hz.

### Synthesis of Key Intermediates

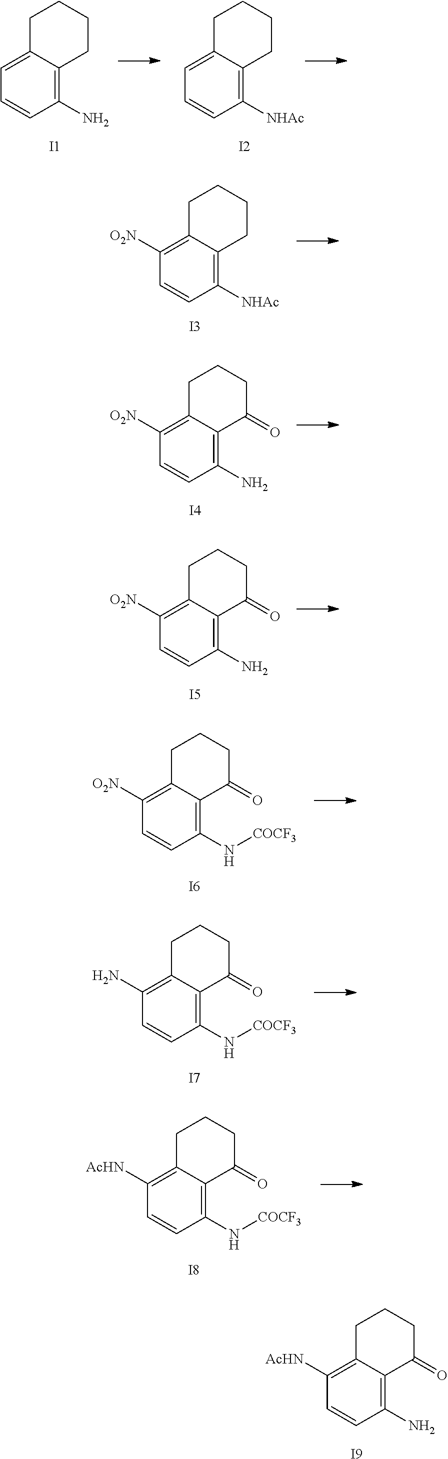

### a) N-(5,6,7,8-tetrahydronaphthalen-1-yl)acetamide (I2)

5,6,7,8-tetrahydronaphthalen-1-amine I1 (8.54 g, 58.0 mmol) was dissolved in dichloromethane (80 mL). Triethylamine (18 mL, 129 mmol) was added and the mixture cooled to 0° C. Dropwise, acetic anhydride (11.5 mL, 122 mmol) was added, upon completion of the addition, the reaction mixture was warmed to rt and stirred for 45 min, whereupon LCMS indicated the reaction was complete. The mixture was diluted with CH₂Cl₂, washed with H₂O, sat. NaHCO₃, 10% citric acid, the organic phase dried over MgSO₄ and concentrated in vacuo. The off-white solid was triturated with 1:3 Et₂O/isohexane to afford 12 (10.8 g, 57.1 mmol, 98% Yield) as a white solid which was used without further purification. LC/MS (method A): retention time 1.44 mins (ES+) m/z 190 [M+H]⁺

### b) N-(4-nitro-5,6,7,8-tetrahydronaphthalen-1-yl)acetamide (13)

N-(5,6,7,8-tetrahydronaphthalen-1-yl)acetamide I2 (1.00 g, 5.2840 mmol) was added portion-wise to sulfuric acid (15 mL, 281 mmol) at −5° C. Sodium nitrate (450 mg, 5.2945 mmol) was added portion-wise to the reaction mixture and stirred for 30 min at −5° C. whereupon LCMS indicated no further reaction progress. The reaction mixture was poured onto ice with external cooling, the aqueous mixture extracted with CH₂Cl₂, the organic phase dried over MgSO₄ and purified by Isolera (10-80% EtOAc in isohexane) to afford a mixture of N-(4-nitro-5,6,7,8-tetrahydronaphthalen-1-yl)acetamide I3 and N-(2-nitro-5,6,7,8-tetrahydronaphthalen-1-yl)acetamide (956 mg, 4.0811 mmol, 77% Yield) as a white/yellow solid. LC/MS (method A): retention time 1.53 mins (ES+) m/z 235 [M+H]⁺.

### c) N-(4-nitro-8-oxo-5,6,7,8-tetrahydronaphthalen-1-yl)acetamide (I4)

N-(4-nitro-5,6,7,8-tetrahydronaphthalen-1-yl)acetamide I3 (1.01 g, 4.31 mmol) was dissolved in acetone (30 mL). Magnesium sulfate in water (3.9 mL, 5.9 mmol, 1.5 mol/L) was added and the mixture was cooled to 0° C. Potassium permanganate (2.07 g, 13.0 mmol) was added portionwise to the reaction mixture and the mixture warmed to rt and stirred for 50 min, whereupon TLC indicated the reaction was complete. The reaction mixture was filtered through Celite, the solids washed with CHCl₃ and the resulting organic mixture washed with H₂O, brine, dried over MgSO₄ and purified by isolera (20-50% EtOAc in isohexane) to afford a mixture of N-(4-nitro-8-oxo-5,6,7,8-tetrahydronaphthalen-1-yl)acetamide I4 and N-(2-nitro-8-oxo-5,6,7,8-tetrahydronaphthalen-1-yl)acetamide (709 mg, 2.86 mmol, 66%) as a white/yellow solid. LC/MS (method A): retention time 1.44 mins (ES+) m/z 190 [M+H]⁺

### d) 8-amino-5-nitro-3,4-dihydronaphthalen-1(2H)-one (I5)

A mixture of N-(4-nitro-8-oxo-5,6,7,8-tetrahydronaphthalen-1-yl)acetamide I4 and N-(2-nitro-8-oxo-5,6,7,8-tetrahydronaphthalen-1-yl)acetamide (709 mg, 2.8561 mmol) and 6N hydrochloric acid (7 mL) were stirred at 80° C. for 2.5 h, whereupon LCMS indicated the reaction was complete. The reaction mixture was cooled in an ice bath and 6N NaOH solution was added until the pH was basic. The aqueous mixture was extracted with CH₂Cl₂, the organic phase dried over MgSO₄ and concentrated in vacuo. Isolera (0-50% EtOAc in isohexane) afforded 8-amino-5-nitro-3,4-dihydronaphthalen-1(2H)-one 15 (320 mg, 1.552 mmol, 54% Yield) as a yellow/orange solid. LC/MS (method A): retention time 1.54 mins (ES+) m/z 207 [M+H]⁺

### e) 2,2,2-trifluoro-N-(4-nitro-8-oxo-5,6,7,8-tetrahydronaphthalen-1-yl)acetamide (I6)

8-amino-5-nitro-3,4-dihydronaphthalen-1(2H)-one I5 (430 mg, 2.0854 mmol) was dissolved in dichloromethane (20 mL). Pyridine (340 μL, 4.20 mmol) was added and the mixture cooled to 0° C. Trifluoroacetic anhydride (590 μL, 4.197 mmol) was added and stirred for 30 min, whereupon LCMS indicated the reaction was complete. The mixture was diluted with CH₂Cl₂, washed with H₂O, the organic phase dried over MgSO₄ and concentrated in vacuo to afford 2,2,2-trifluoro-N-(4-nitro-8-oxo-5,6,7,8-tetrahydronaphthalen-1-yl)acetamide I6 (630 mg, 2.0846 mmol, >99% Yield) as a yellow solid, which was used without further purification. LC/MS (method A): retention time 1.86 min (ES+) m/z 301X [M−H]⁻

### f) N-(4-amino-8-oxo-5,6,7,8-tetrahydronaphthalen-1-yl)-2,2,2-trifluoroacetamide (I7)

Zinc (2.73 g, 41.7 mmol) was suspended in methanol (80 mL), formic acid (4 mL) and water (4 mL) and the mixture cooled to 0° C. 2,2,2-trifluoro-N-(4-nitro-8-oxo-5,6,7,8-tetrahydronaphthalen-1-yl)acetamide I6 (568 mg, 2.0865 mmol) was added portion-wise and the mixture stirred at 0° C. for 30 min, whereupon LCMS indicated the reaction was complete. The reaction mixture was filtered, the filtrate diluted with EtOAc and washed with sat NaHCO₃. The organic phase was dried over MgSO₄ and concentrated in vacuo to afford N-(4-amino-8-oxo-5,6,7,8-tetrahydronaphthalen-1-yl)-2,2,2-trifluoroacetamide 17 (568 mg, 2.0865 mmol, >99% Yield) as a yellow solid, which was used without further purification. LC/MS (method A): retention time 1.65 min (ES+) m/z 273 [M+H]⁺

### g) N-(4-acetamido-8-oxo-5,6,7,8-tetrahydronaphthalen-1-yl)-2,2,2-trifluoroacetamide (I8)

N-(8-amino-4-oxo-tetralin-5-yl)-2,2,2-trifluoro-acetamide I7 (568 mg, 2.0865 mmol) was dissolved in dichloromethane (20 mL). Triethylamine (580 μL, 4.16 mmol) then acetyl chloride (297 μL, 4.173 mmol) were added and the mixture stirred for 30 min, whereupon LCMS indicated the reaction was complete. The reaction mixture was diluted with CH₂Cl₂, washed with H₂O, the organic phase dried over MgSO₄ and concentrated in vacuo to afford N-(8-acetamido-4-oxo-tetralin-5-yl)-2,2,2-trifluoro-acetamide I8 (655 mg, 2.084 mmol, >99% yield) as a yellow solid, which was used without further purification. LC/MS (method A): retention time 1.55 min (ES+) m/z 315 [M+H]⁺

### h) N-(4-amino-5-oxo-5,6,7,8-tetrahydronaphthalen-1-yl)acetamide (I9)

N-(8-acetamido-4-oxo-tetralin-5-yl)-2,2,2-trifluoro-acetamide I8 (2.77 g, 8.81 mmol) was dissolved in methanol (240 mL) and water (17 mL). Potassium carbonate (4.88 g, 35.3 mmol) was added and the mixture stirred for 1.5 h at 50° C., whereupon LCMS indicated the reaction was complete. The reaction mixture was cooled, concentrated in vacuo, dissolved in 10% MeOH in CH₂Cl₂ and washed with H₂O. The organic phase was dried over MgSO₄ and purified by isolera chromatography (2-15% MeOH in CH₂Cl₂) to afford N-(8-amino-1-oxo-tetralin-5-yl)acetamide 19 (1.20 g, 5.50 mmol, 62.3% Yield) as a yellow solid. LC/MS (method A): retention time 0.98 min (ES+) m/z 219 [M+H]⁺

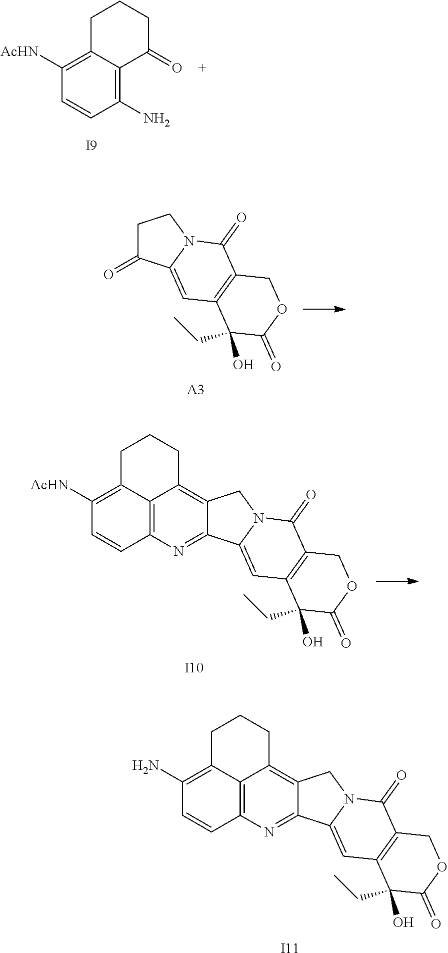

### i) (S)—N-(9-ethyl-9-hydroxy-10,13-dioxo-2,3,9,10,13,15-hexahydro-1H, 12H-benzo[de]pyrano[3,4′:6,7]indolizino[1,2-b]quinolin-4-yl)acetamide (I10)

N-(8-amino-1-oxo-tetralin-5-yl)acetamide I9 (641 mg, 2.94 mmol, 1.0 eq.), (S)-4-ethyl-4-hydroxy-7,8-dihydro-1H-pyrano[3,4-f]indolizine-3,6,10(4H)-trione A3 (840 mg, 3.19 mmol, 1.1 eq.) and PPTS (740 mg, 2.95 mmol, 1.0 eq.) were dissolved in toluene (60 mL) and stirred at reflux for 3 h, whereupon LCMS indicated 19 had been consumed. The reaction mixture was cooled and concentrated in vacuo. The resulting solids were triturated with acetonitrile, then acetone to afford 110 as a brown solid with minor TsOH contamination (1.26 g, 96%). LC/MS (method A): retention time 1.32 mins (ES+) m/z 447 [M+H]⁺

### j) (S)-4-amino-9-ethyl-9-hydroxy-1,2,3,9,12,15-hexahydro-10H, 13H-benzo[de]pyrano[3,4′:6,7]indolizino[1,2-b]quinoline-10,13-dione (I11)

(S)—N-(9-ethyl-9-hydroxy-10, 13-dioxo-2,3,9, 10,13, 15-hexahydro-1H, 12H-benzo[de]pyrano[3′,4′:6,7]indolizino[1,2-b]quinolin-4-yl)acetamide (I10) (1.26 g, 2.83 mmol, 1.0 eq.) was dissolved in hydrochloric acid (6 mol/L) in H₂O (12 mL) and the mixture stirred for 5 h at 80° C., whereupon LCMS indicated 110 had been consumed. The reaction mixture was diluted with H₂O and concentrated in vacuo to afford (S)-4-amino-9-ethyl-9-hydroxy-1,2,3,9,12,15-hexahydro-10H, 13H-benzo[de]pyrano[3′,4′:6,7]indolizino[1,2-b]quinoline-10,13-dione I11 (1.51 g, 2.85 mmol, 90 mass %, 101% Yield) as a red crystaline solid. LC/MS (method A): retention time 1.36 mins (ES+) m/z 405 [M+H]⁺.

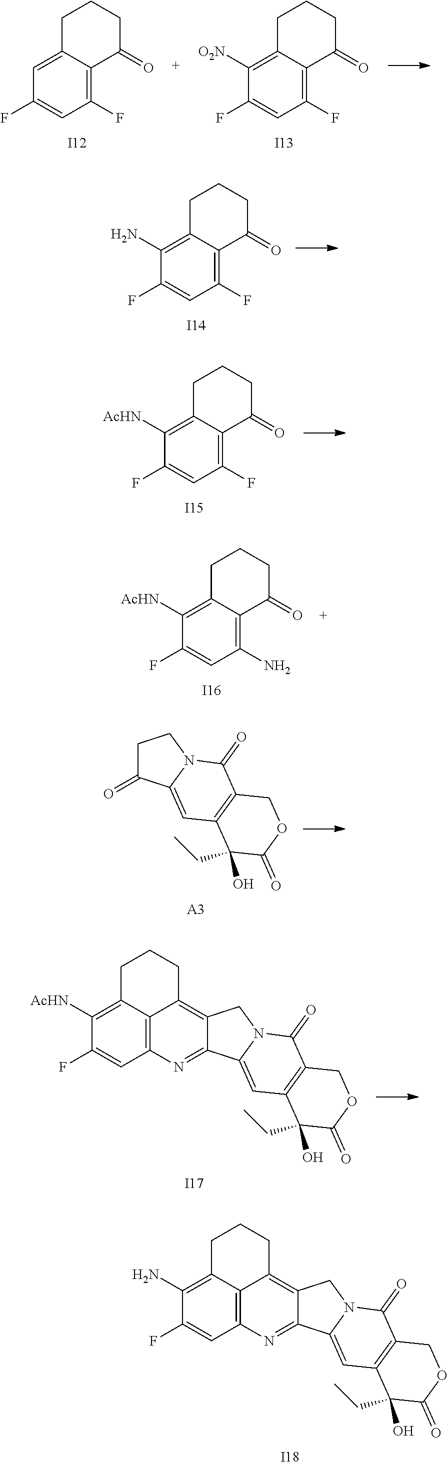

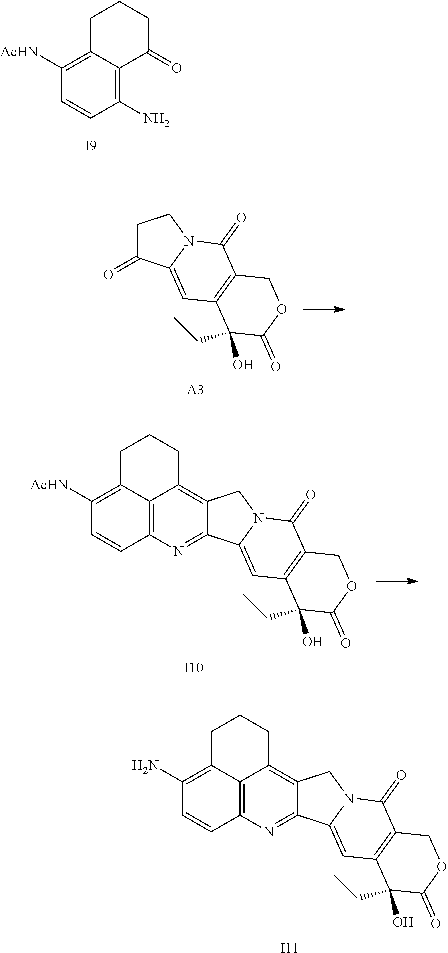

### a) 6,8-Difluoro-5-nitro-1-tetralone (I13)

To a dust of 6,8-difluoro-1-tetralone I12 (15 g, 82.3 mmol) was added dropwise concentrated H₂SO₄ (90 mL) at 0° C. To the resulting mixture was added KNOB (8.2 g, 90.1 mmol) in portion-wise at 0° C. The reaction mixture was stirred at 0° C. for 2 h. The reaction was quenched with ice-water (200 mL) and then extracted with EtOAc (400 mL×3). The combined organic layers were washed with aqueous NaHCO₃ (400 mL) and brine (400 mL), dried over anhydrous MgSO₄ and concentrated under reduced pressure. The residue was purified by column chromatography on silica gel (petroleum ether/EtOAc=100:1) to afford compound I13 (8.1 g, 43% yield). ¹H NMR (400 MHz, CDCl₃): δ ppm 6.98 (t, J=10.0 Hz, 1 H), 3.01-2.98 (m, 2H), 2.72-2.68 (m, 2H), 2.21-2.05 (m, 2H).

### b) 5-Amino-6,8-difluoro-1-tetralone (I14)

To a mixture of compound I13 (9.1 g, 39.6 mmol) in EtOH/H₂O (8:1, 270 mL) were added NH₄Cl (6.4 g, 0.12 mol) and dust Fe (17.6 g, 0.32 mol). The reaction mixture was stirred at 80° C. for 2 h. The reaction mixture was cooled to room temperature and filtered. The filtrate was concentrated under reduced pressure. The residue was diluted with water (50 mL) and then extracted with EtOAc (200 mL×3). The combined organic layers were washed with brine (200 mL), dried over anhydrous MgSO₄ and concentrated under reduced pressure. The residue was purified by column chromatography on silica gel (petroleum ether/EtOAc=8:1) to afford compound I14 (7.3 g, 94% yield). ¹H NMR (400 MHz, DMSO-d₆): δ ppm 7.04 (t, J=11.6 Hz, 1H), 5.05 (br s, 2H), 2.71-2.2.68 (m, 2H), 2.5 (m, 2H), 2.03-1.98 (m, 2H).

### c) 5-Acetylamino-6,8-difluoro-1-tetralone (I15)

To a solution of compound I14 (7.3 g, 37 mmol) and Et₃N (4.5 g, 44.4 mmol) in DCM (100 mL) was added dropwise Ac₂O (4.5 g, 44.4 mmol) at room temperature. The reaction mixture was stirred at room temperature overnight. The mixture was concentrated under reduced pressure. The residue was purified by column chromatography on silica gel (DCM/MeOH=300:1) to afford compound I15 (5.3 g, 60% yield). ¹H NMR (400 MHz, CDCl₃): δ ppm 6.84 (t, J=10 Hz, 1H), 6.75 (br s, 1H), 2.89-2.86 (m, 2H), 2.66-2.63 (m, 2H), 2.25 (s, 3H), 2.10-2.06 (m, 2H).

### d) 5-Acetylamino-6-fluoro-8-amino-1-tetralone (I16)

To a solution of compound I15 (5.2 g, 21.7 mmol) in DMSO (50 mL) was added 25% aqueous NH₄OH (80 mL) at room temperature. The reaction mixture was stirred at 130° C. for 16 h. The mixture was cooled to room temperature and then extracted with EtOAc (200 mL×5). The combined organic layers were washed with brine (200 mL), dried over anhydrous MgSO₄ and concentrated under reduced pressure. The residue was purified by column chromatography on silica gel (DCM/MeOH=100:1) to afford compound I16 (1.5 g, 30% yield) as a brownish solid. ¹H NMR (400 MHz, DMSO-d₆): δ ppm 9.16 (s, 1H), 6.42 (d, J=12.4 Hz, 1H), 2.66 (m, 2H), 2.55-2.48 (m, 2H), 2.00 (s, 3H), 1.88-1.85 (m, 2H).

### e) (S)—N-(9-ethyl-5-fluoro-9-hydroxy-10,13-dioxo-2,3,9,10,13,15-hexahydro-1H,12H-benzo[de]pyrano[3′,4′:6,7]indolizino[1,2-b]quinolin-4-yl)acetamide I17

Compound I16 (150 mg, 0.635 mmol), 168 mg (0.638 mmol) of (4S)-4-ethyl-4-hydroxy-7,8-dihydro-1H-pyrano[3,4-f]indolizine-3,6,10-trione A3, and 168 mg (0.668 mmol) of pyridinium p-toluenesulfonate were mixed in 30 mL of anhydrous toluene. Equipped with a Dean-Stark trap, the reaction was heated with at 130° C. for 4 h. There was a water layer in the condenser. The solvent was evaporated, and the residue was precipitated into 14 mL of acetone and centrifuged to get 180 mg of the desired product as a brown solid. The residue on the flask wall was washed off with acetone and collected to give 60 mg of the desired product as a brown solid. The combined yield of the crude product 117 was 82%. LCMS (0.1% formic acid/acetonitrile) ESI [M+H]=464; ¹H NMR (400 MHZ, DMSO-d₆): signals for the desired product, δ ppm 9.77 (s, 1H), 7.72 (d, J=11.1 Hz, 1H), 7.25 (s, 1H), 5.36 (s, 2H), 5.17 (s, 2H), 3.09 (t, J=5.5 Hz, 2H), 2.91 (t, J=5.5 Hz, 2H), 2.22 (s, 1H), 2.08 (s, 3H), 1.96 (m, 2H), 1.80 (m, 2H), 0.81 (t, J=7.3 Hz, 3H).

### f) (S)-4-amino-9-ethyl-5-fluoro-9-hydroxy-1,2,3,9,12,15-hexahydro-10H,13H-benzo[de]pyrano[3′,4′:6,7]indolizino[1,2-b]quinoline-10,13-dione I18

60 mg of crude compound I17 was dissolved in 0.5 mL of HCl (37%), and the reaction was carried out in a sealed tube in a microwave reactor at 100° C. for 1 h. The solvent was evaporated, and the residue was dissolved in 1 mL of NMP and purified on Prep-HPLC with 0.1% TFA in water as A solvent and 0.1% TFA in acetonitrile as B solvent. The fractions containing the desired product were collected and frozen. After lyophilization, the reaction afforded 28 mg (42%) of the desired product 118 as an orange solid. LCMS (0.1% formic acid/acetonitrile) ESI [M+H]=422; ¹H NMR (400 MHz, DMSO-d₆): δ ppm 7.56 (d, J=12.4 Hz, 1H), 7.14 (s, 1H), 5.34 (s, 2H), 5.10 (s, 2H), 2.99 (t, J=6.1 Hz, 2H), 2.78 (t, J=6.1 Hz, 2H), 1.95 (t, J=5.8 Hz, 2H), 1.79 (m, 2H), 1.40-1.00 (m, 3H), 0.81 (t, J=7.4 Hz, 3H).

### Example 1

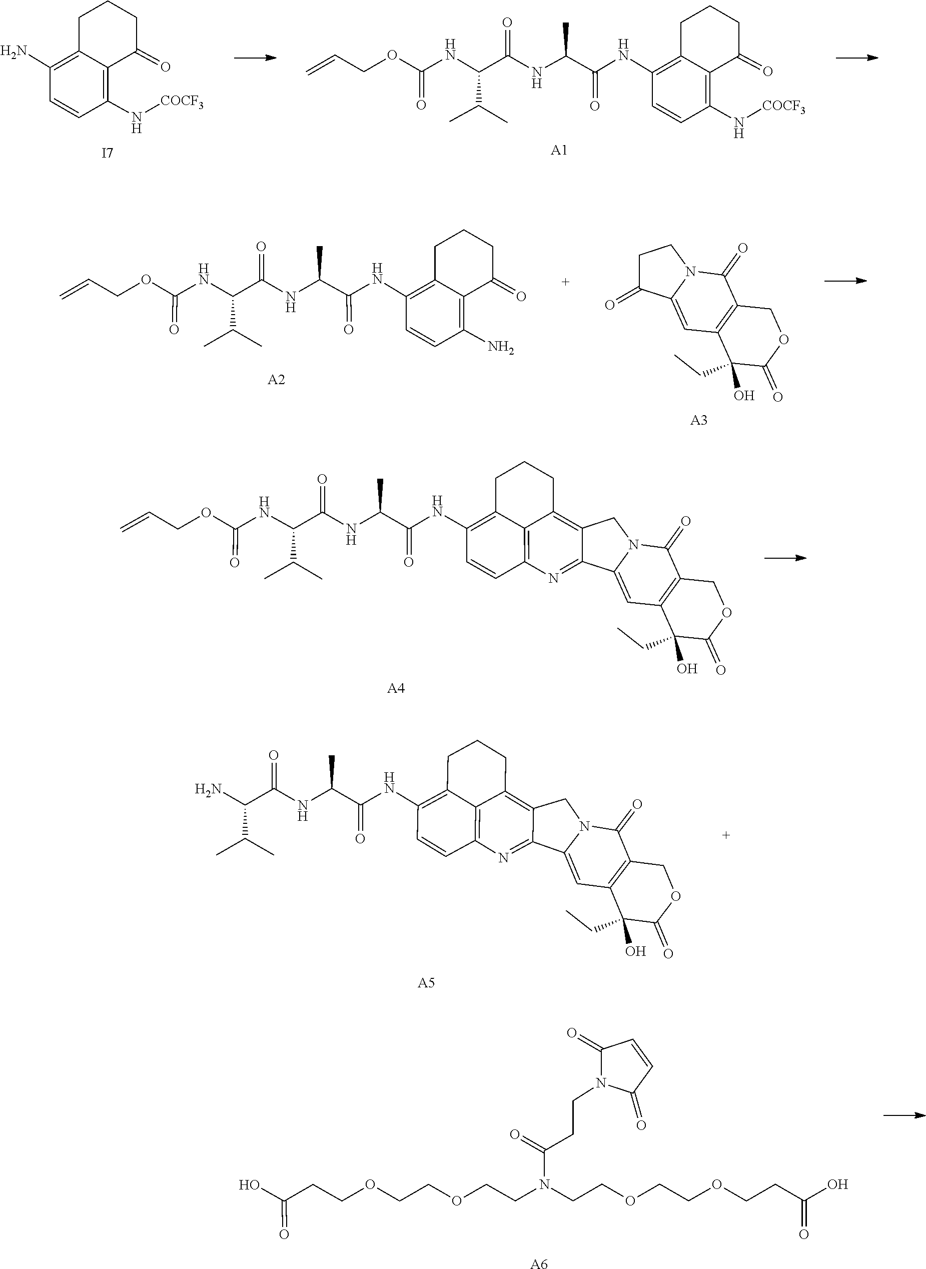

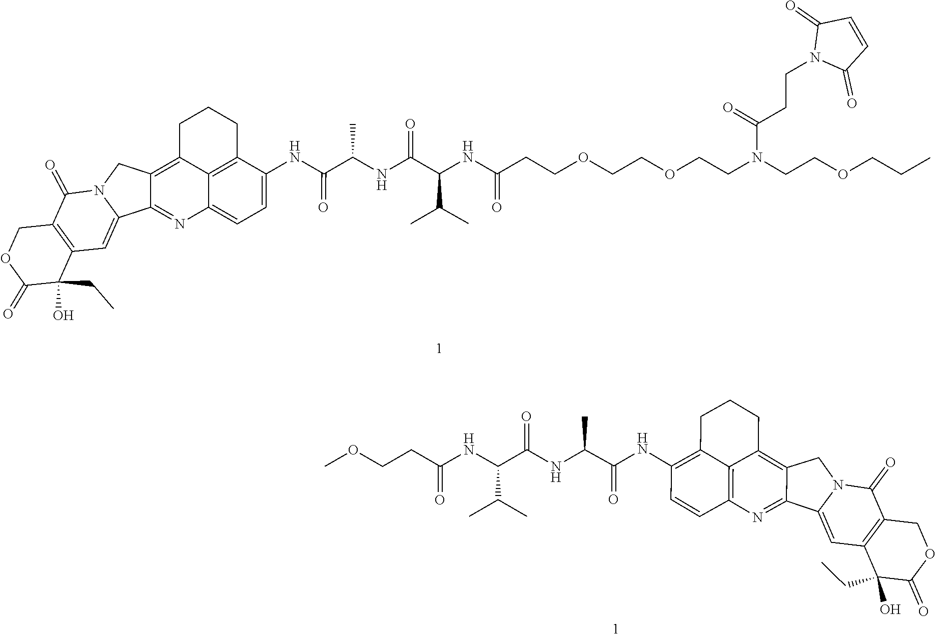

### a) Allyl ((S)-3-methyl-1-oxo-1-(((S)-1-oxo-((5-oxo-4-(2,2,2-trifluoroacetamido)-5,6,7,8-tetrahydronaphthalen-1-yl)amino)propan-2-yl)amino)butan-2-yl)carbamate (A1)

DCC (6.54 g, 31.7 mMol) and HOPO (3.36 g, 30.2 mMol) were added to a solution of alloc-Val-Ala-OH (9.09 g, 31.7 mmol) and 17 (7.85 g, 28.8 mMol) in CH₂Cl₂ (300 mL) at 25° C.. The resulting mixture was left to stir overnight. The white solid that formed during the reaction was filtered out and washed with cold CH₂Cl₂. The filtrate was washed with water (150 mL) and brine (150 mL). The organic layer was dried over MgSO₄, filtered and evaporated. The crude product was purified by silica gel chromatography (Hex/EtOAc, 60:40). Product A1 isolated was contaminated with co-eluting DCU (21.1 g, 140% yield). LC/MS (Method B): ES⁺=1.81 min, m/z 527.6 [M+H]⁺.

### b) Allyl ((S)-1-(((S)-1-((4-amino-5-oxo-5,6,7,8-tetrahydronaphthalen-1-yl)amino)-1-oxopropan-2-yl)amino)-3-methyl-1-oxobutan-2-yl)carbamate (A2)

Protected aniline A1 (18 g, 34.19 mMol) was solubilised in a mixture of MeOH and H₂O 10:1 (165 mL) and K₂CO₃ was added (10 g, 72.36 mMol). The mixture was stirred at 50° C. until complete. The mixture was vacced down to almost dryness and the residue was taken up with CH₂Cl₂ and washed with H₂O and brine, before being dried over MgSO₄, filtered and evaporated. The crude product was purified by silica gel chromatography (CHCl₃/MeOH, 100% to 7:3). The isolated product A2 was contaminated with a co-eluting impurity (10.71 g, 73% yield). LC/MS (Method B): ES⁺=1.46 min, m/z 431.7 [M+H]⁺.

### c) Allyl ((S)-1-(((S)-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-1-oxopropan-2-yl)amino)-3-methylbutan-2-yl)carbamate (A4)

Aniline A2 (450 mg, 1.045 mMol), lactone A3 (280 mg, 1.064 mMol) and pyridinium p-toluenesulfonate (273 mg, 1.086 mMol) were solubilised in toluene (20 mL) and the mixture was heated to 130° C. (high reflux). Every now and then a few drops of MeOH is added to help solubilise the mixture. After 7h the crude reaction was vacced down to dryness. The crude product was purified by silica gel chromatography (CHCl₃/MeOH, 100% to 95:5) to give product A4 (360 mg, 52.3% yield). LC/MS (Method B): ES⁺=1.51 min, m/z 658.8 [M+H]⁺.

### d) Allyl (S)-2-amino-N—((S)-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-1-oxopropan-2-yl)-3-methylbutanamide (A5)

Excess piperidine was added (642 μL) to a solution of A4 (543 mg, 0.82 mMol) and PdP(Ph₃)₄ (89 mg, 0.08 mMol) in CH₂Cl₂ (15 mL). The mixture was allowed to stir at room temperature for 20 min, at which point the reaction had gone to completion (as monitored by LC/MS). The reaction mixture was diluted with CH₂Cl₂ (25 mL) and the organic phase was washed with H₂O (25 mL) and brine (25 mL). The organic phase was dried over MgSO₄, filtered and excess solvent removed by rotary evaporation under reduced pressure to afford crude product A5 which was used as such in the next step. LC/MS (Method B): ES⁺=1.15 min, m/z 574.6 [M+H]⁺.

### e) (S)-2-((2S,5S)-16-(3-(2,5-dioxo-2,5-dihydro-1H-pyrrol-1-yl)propanoyl)-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-5-isopropyl-2-methyl-1,4,7-trioxo-10,13,19,22-tetraoxa-3,6,16-triazapentacosan-25-amido)-N—((S)-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-1-oxopropan-2-yl)-3-methylbutanamide (1)

EDCI.HCl (0.26 mmol, 2.1 eq) was added to a solution of A6 (purchased from Broadpharm) (0.122 mmol, 1.0 eq) in DCM (25 mL) and the resulting mixture stirred at room temperature for 60 min. A5 (0.26 mmol, 2.1 eq) was added and stirring continued for a further 2 hrs. The reaction mixture was evaporated to dryness and the residue purified by prep HPLC (30-40% MeCN/water+0.05% formic acid over 10 mins) to leave the product 1 as a white solid. Yield=23 mg (12%). LC/MS (Method B): rt 1.54 min m/z 1601.2 [M+H]⁺.

### Example 2—Conjugation

Herceptin-C239i antibody

Herceptin antibodies were engineered to have cysteine inserted between the 239 and 240 positions were produced following the methods described in Dimasi, N., et al., Molecular Pharmaceutics, 2017, 14, 1501-1516 (DOI: 5 10.1021/acs.molpharmaceut.6b00995).

A 50 mM solution of (TCEP) in phosphate-buffered saline pH 7.4 (PBS) was added (40 molar equivalent/antibody, 2.67 micromoles, 53.3 μL) to a 3.3 mL solution of Herception-C239i antibody (10 mg, 67 nanomoles) in reduction buffer containing PBS and 1 mM ethylenediaminetetraacetic acid (EDTA) and a final antibody concentration of 3.0 mg/mL. The reduction mixture was allowed to react at room temperature for 17 hours (or until full reduction is observed by UHPLC) in an orbital shaker with gentle (60 rpm) shaking. The reduced antibody was buffer exchanged, via spin filter centrifugation, into a reoxidation buffer containing 30 mM Histidine, 30 mM Arginine pH 6.8 and 1 mM EDTA to remove all the excess reducing agent. A 50 mM solution of dehydroascorbic acid (DHAA, 30 molar equivalent/antibody, 2.0 micromoles, 40 μL) in DMSO was added and the reoxidation mixture was allowed to react for 3 hours at room temperature with gentle (60 rpm) shaking at an antibody concentration of 3.0 mg/mL (or more DHAA added and reaction left for longer until full reoxidation of the cysteine thiols to reform the inter-chain cysteine disulfides is observed by UHPLC). The reoxidation mixture was then sterile-filtered and diluted in a conjugation buffer containing PBS and 1 mM EDTA for a final antibody concentration of 2.0 mg/mL. Compound 1 was added as a DMSO solution (10 molar equivalent/antibody, 0.5 micromole, in 0.375 mL DMSO) to 3.375 mL of this reoxidised antibody solution (7.5 mg, 50 nanomoles) for a 10% (v/v) final DMSO concentration. The solution left to react at room temperature for 2 hours at room temperature with gentle shaking. Then the conjugation was quenched by addition of N-acetyl cysteine (2.5 micromoles, 25 μL at 100 mM), then purified on an AKTA™ Start FPLC using a GE Healthcare HiLoad™ 26/600 column packed with Superdex 200 PG, eluting with 2.6 mL/min PBS. Fractions corresponding to ConjA monomer peak were pooled, concentrated using a 15 mL Amicon Ultracell 30 kDa MWCO spin filter, sterile-filtered and analysed.

UHPLC analysis on a Shimadzu Prominence system using a Thermo Scientific MAbPac 50 mm×2.1 mm column eluting with a gradient of water and acetonitrile on a reduced sample of ConjA at 214 nm and 330 nm (Compound 1 specific) shows unconjugated light chains and a mixture of unconjugated heavy chains and heavy chains attached to a single molecule of Compound 1, consistent with a drug-per-antibody ratio (DAR) of 3.88 molecules of Compound 1 per antibody (since each molecule of Compound 1 contains two drugs).

UHPLC analysis on a Shimadzu Prominence system using a Tosoh Bioscience TSKgel SuperSW mAb HTP 4 μm 4.6×150 mm column (with a 4 μm 3.0×20 mm guard column) eluting with 0.3 mL/minute sterile-filtered SEC buffer containing 200 mM potassium phosphate pH 6.95, 250 mM potassium chloride and 10% isopropanol (v/v) on a sample of ConjA at 280 nm shows a monomer purity of 98%. UHPLC SEC analysis gives a concentration of final ConjA at 2.13 mg/mL in 2.5 mL, obtained mass of ConjA is 5.32 mg (71% yield).

### Example 3—ADC In Vitro Assay

The concentration and viability of cells from a sub-confluent (80-90% confluency) T75 flask are measured by trypan blue staining, and counted using the LUNA-II™ Automated Cell Counter. Cells were diluted to 2×10⁵/ml, dispensed (50 pl per well) into 96-well flat-bottom plates.

A stock solution (1 ml) of antibody drug conjugate (ADC) (20 μg/ml) was made by dilution of filter-sterilised ADC into cell culture medium. A set of 8x 10-fold dilutions of stock ADC were made in a 24-well plate by serial transfer of 100 pl into 900 pl of cell culture medium. ADC dilution was dispensed (50 pl per well) into 4 replicate wells of the 96-well plate, containing 50 pl cell suspension seeded the day previously. Control wells received 50 pl cell culture medium. The 96-well plate containing cells and ADCs was incubated at 37° C. in a CO2-gassed incubator for the exposure time.

At the end of the incubation period, cell viability was measured by MTS assay. MTS (Promega) was dispensed (20 pl per well) into each well and incubated for 4 hours at 37° C. in the CO₂-gassed incubator. Well absorbance was measured at 490 nm. Percentage cell survival was calculated from the mean absorbance in the 4 ADC-treated wells compared to the mean absorbance in the 4 control untreated wells (100%). IC₅₀ was determined from the dose-response data using GraphPad Prism using the non-linear curve fit algorithm: sigmoidal dose-response curve with variable slope.

ADC incubation times were 4 days with MDA-MB-468 and 7 days for NCI-N87. MDA-MB-468 and NCI-N87 were cultured in RPMI 1640 with Glutamax+10% (v/v) HyClone™ Fetal Bovine Serum.

| EC₅₀ (μg/mL) | NCI-N87 | MDA-MB-468 |
|---|---|---|
| ConjA | 0.0492 | >10 |

### STATEMENTS OF INVENTION

1. A compound with the formula I:

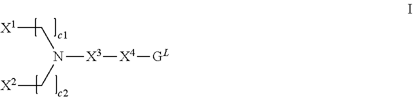

where X¹ and X² are independently selected from a group of formula Ia:

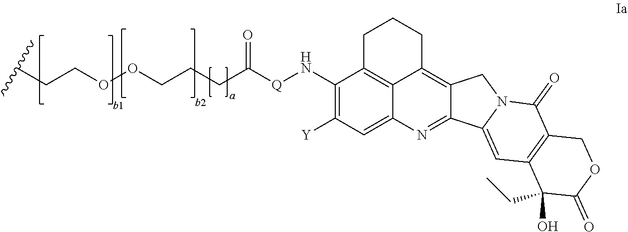

Q is:

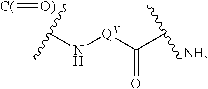

where Q^{X} is such that Q is an amino-acid residue, a dipeptide residue, a tripeptide residue or a tetrapeptide residue;

a=0 to 5, b1=0 to 16 and b2=0 to 16, wherein at least b1 or b2=0 (i.e. only one of b1 and b2 may not be 0);

Y is H or F;

c1 is 0 to 5;

c2 is 0 to 5;

X³ is —CH₂— or —C(═O)—;

X⁴ is ^{X3}—(CH₂)_{d1}—(C₂H₄O)ₑ—(CH₂)_{d2}—^{GL}, where d1 is 0 to 5, d2 is 0 to 5 and e is 0 to 16;

and

G^{L} is a linker for connecting to a Ligand Unit.

2. The compound according to statement 1, wherein Q is an amino acid residue.

3. The compound according to statement 2, wherein Q is selected from: Phe, Lys, Val, Ala, Cit, Leu, Ile, Arg, and Trp.

4. The compound according to statement 1, wherein Q is a dipeptide residue.

5. The compound according to statement 4, wherein Q is selected from:
_{NH}-Phe-Lys-^{C═O},
^{NH}-Val-Ala-^{C═O},
^{NH}-Val-Lys-^{C═O},
^{NH}-Ala-Lys-^{C═O},
^{NH}-Val-Cit-^{C═O},
^{NH}-Phe-Cit-^{C═O},
^{NH}-Leu-Cit-^{C═O},
^{NH}-Ile-Cit-^{C═O},
^{NH}-Phe-Arg-^{C═O},
^{NH}-Trp-Cit-^{C═O}, and
^{NH}-Gly-Val-^{C═O}.

6. The compound according to statement 5, wherein Q is selected from ^{NH}-Phe-Lys-^{C═O}, ^{NH}-Val-Cit-^{C═O} and ^{NH}-Val-Ala-^{C═O}.

7 The compound according to statement 1, wherein Q is a tripeptide residue.

8. The compound according to statement 7, wherein Q is selected from ^{NH}-Glu-Val-Ala-^{C═O},^{NH}-Glu-Val-Cit-^{C═O}, ^{NH}-αGlu-Val-Ala-^{C═O} and ^{NH}-αGlu-Val-Cit-^{C═O}.

9. The compound according to statement 1, wherein Q is a tetrapeptide residue.

10. The compound according to statement 9, wherein Q is selected from:
^{NH}-Gly-Gly-Phe-Gly ^{C═O}; and
^{NH}-Gly-Phe-Gly-Gly ^{C═O}.

11. The compound according to statement 10, wherein Q is:
^{NH}-Gly-Gly-Phe-Gly ^{C═O}.

12. The compound according to any one of statements 1 to 11, wherein a is 0 to 3.

13. The compound according to statement 12, wherein a is 0 or 1.

14. The compound according to statement 12, wherein a is 0.

15. The compound according to any one of statements 1 to 14, wherein b1 is 0 to 8.

16. The compound according to statement 15, wherein b1 is 0.

17. The compound according to statement 15, wherein b1 is 2.

18. The compound according to statement 15, wherein b1 is 3

19. The compound according to statement 15, wherein b1 is 4.

20. The compound according to statement 15, wherein b1 is 5.

21. The compound according to statement 15, wherein b1 is 8.

22. The compound according to any one of statements 1 to 14 and 16, wherein b2 is 0 to 8.

23. The compound according to statement 22, wherein b2 is 0.

24. The compound according to statement 22, wherein b2 is 2.

25. The compound according to statement 22, wherein b2 is 3.

26. The compound according to statement 22, wherein b2 is 4.

27. The compound according to statement 22, wherein b2 is 5.

28. The compound according to statement 22, wherein b2 is 8.

29. The compound according to any one of statements 1 to 28, wherein Y is H.

30. The compound according to any one of statements 1 to 28, wherein Y is F.

31. The compound according to any one of statements 1 to 30, wherein X¹ and X² are the same.

32. The compound according to any one of statements 1 to 31, wherein c1 is 0 to 3.

33. The compound according to statement 32, wherein c1 is 1 or 2.

34. The compound according to statement 33, wherein c1 is 2.

35. The compound according to any one of statements 1 to 34, wherein c2 is 0 to 3.

36. The compound according to statement 35, wherein c2 is 1 or 2.

37. The compound according to statement 36, wherein c2 is 2.

38. The compound according to any one of statements 1 to 34, wherein c1 and c2 are the same.

39. The compound according to any one of statements 1 to 38, wherein X³ is —CH₂—.

40. The compound according to any one of statements 1 to 38, wherein X³ is —C(═O)—.

41. The compound according to any one of statements 1 to 40, wherein d1 is 0 to 3.

42. The compound according to statement 41, wherein d1 is 1 or 2.

43. The compound according to statement 42, wherein d1 is 2.

44. The compound according to any one of statements 1 to 43, wherein d2 is 0 to 3.

45. The compound according to statement 44, wherein d2 is 1 or 2.

46. The compound according to statement 44, wherein d2 is 0.

47. The compound according to any one of statements 1 to 40, wherein d1+d2 is 0 to 3.

48. The compound according to statement 47, wherein d1+d2 is 2.

49. The compound according to any one of statements 1 to 48, wherein e is 0 to 8.

50. The compound according to statement 49, wherein e is 0.

51. The compound according to statement 49, wherein e is 2.

52. The compound according to statement 49, wherein e is 4.

53. The compound according to statement 49, wherein e is 8.

54. The compound according to any one of statements 1 to 53, wherein e+the largest value of b1 or b2 is no more than 16.

55. The compound according to statement 54, wherein e+the largest value of b1 or b2 is no more than 8.

56. The compound according to any one of statements 1 to 55, wherein each a is 0, each b1 is 0, each b2 is 2, c1 is 2, c2 is 2, X³=—C(═O)—, d1 is 2, d2 is 0 and e is 0.

57. The compound according to any one of statements 1 to 56, wherein G^{L} is selected from

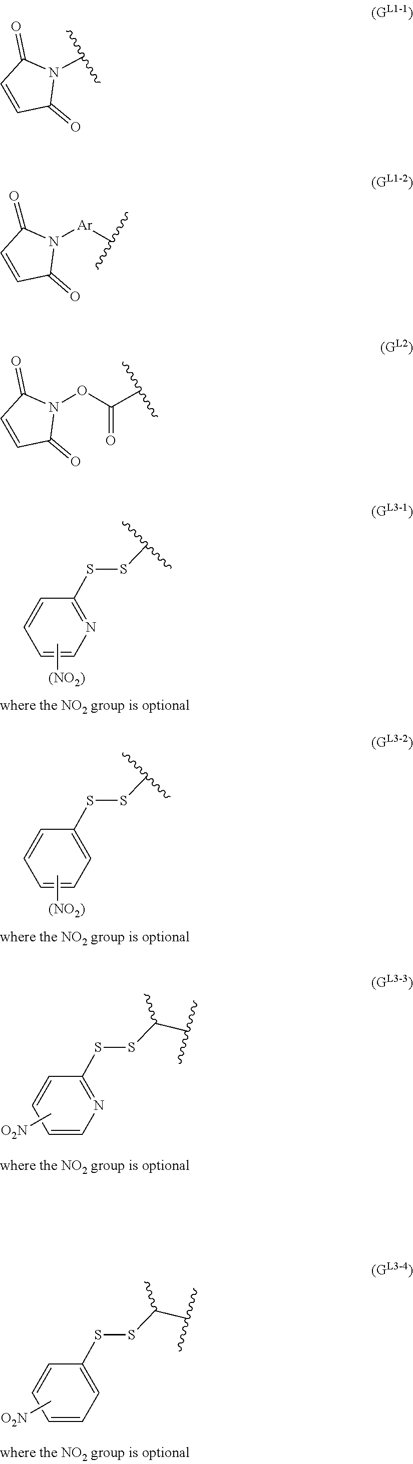

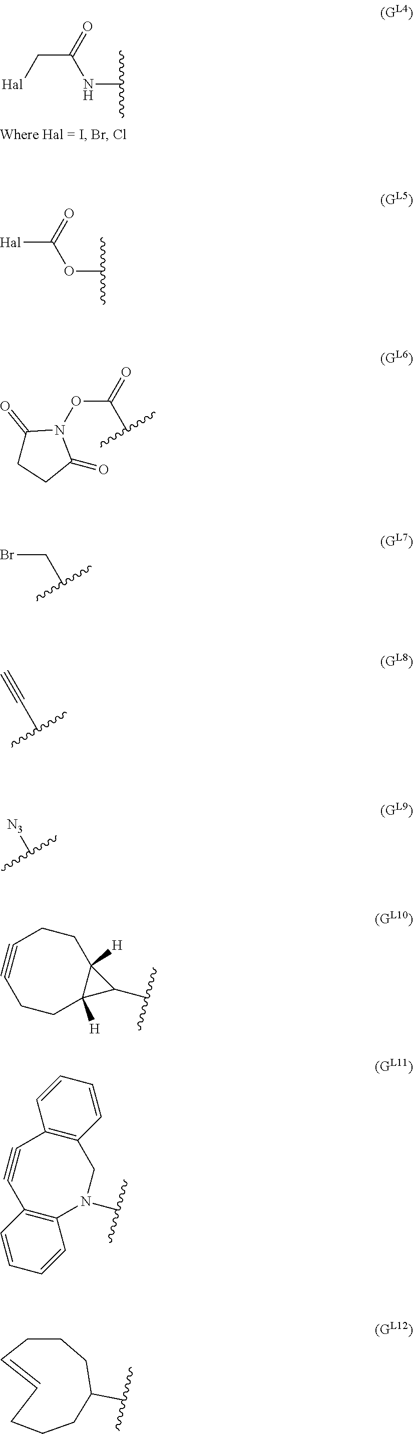

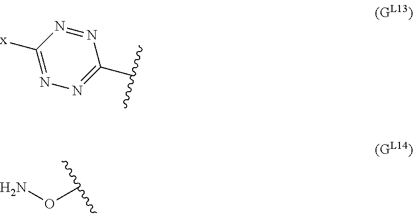

where Ar represents a C₅₋₆ arylene group, and X represents C₁₋₄ alkyl.

58. A compound according to statement 57, wherein G^{L} is selected from G^{L1-1} and G^{L1-2.}

59. A compound according to statement 57, wherein G^{L} is G^{L1-1}.

60. A conjugate of formula IV:

L−(D^{L})_{P} (IV)

or a pharmaceutically acceptable salt or solvate thereof, wherein L is a Ligand unit (i.e., a targeting agent), D^{L} is a Drug Linker unit that is of formula III:

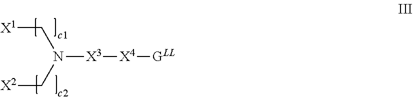

where X¹, X², X³, X⁴, c1 and c2 are as defined in any one of statements 1 to 56;

G^{LL} is a linker connected to a Ligand Unit; and

p is an integer of from 1 to 20.

61. The conjugate according to statement 60, wherein G^{LL} is selected from:

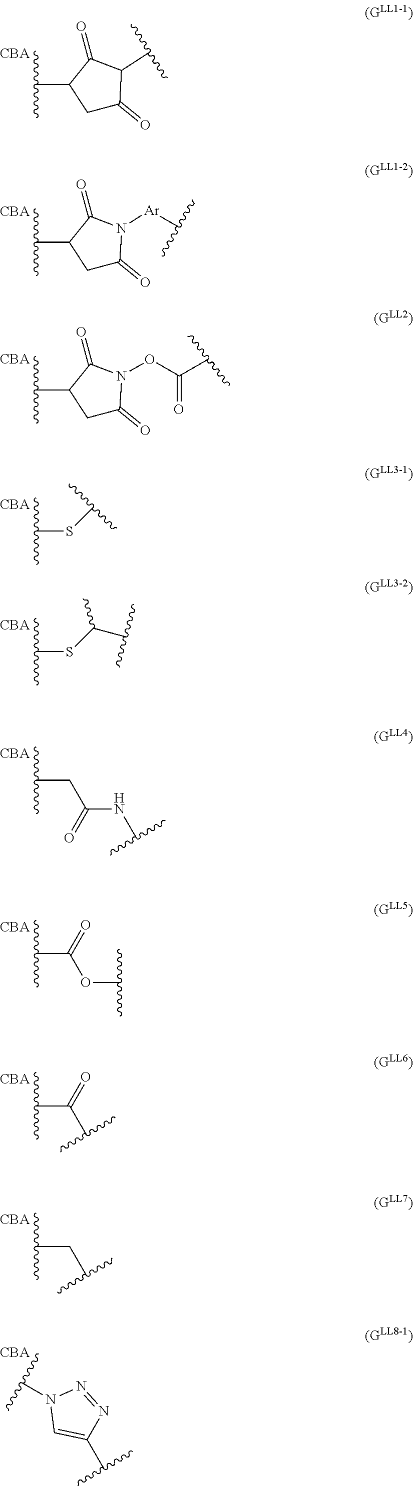

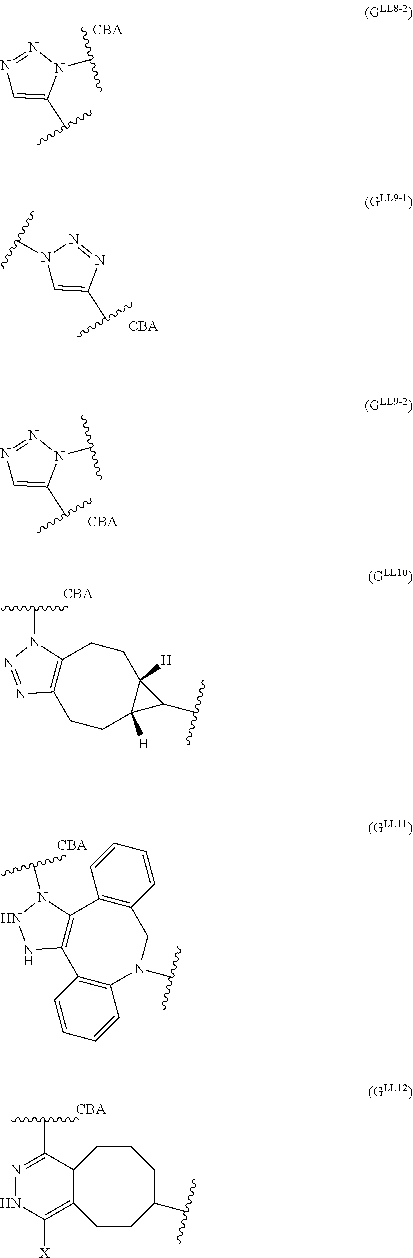

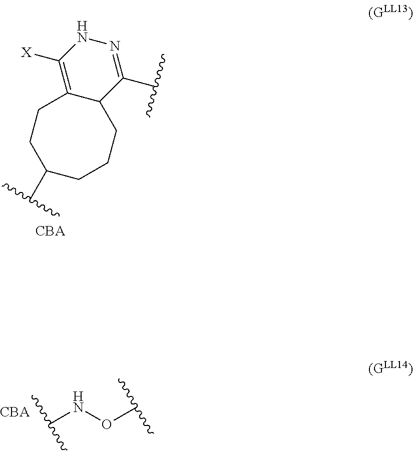

where Ar represents a C₅₋₆ arylene group and X represents C₁₋₄ alkyl.

62. The conjugate according to statement 61, wherein G^{LL} is selected from G^{LL1-1} and G^{LL1-2}.

63. The conjugate according to statement 62, wherein G^{LL} is G^{LL1-1}.

64. The conjugate according to any one of statements 60 to 63, wherein the Ligand Unit is an antibody or an active fragment thereof.

65. The conjugate according to statement 64, wherein the antibody or antibody fragment is an antibody or antibody fragment for a tumour-associated antigen.

66. The conjugate according to statement 65, wherein the antibody or antibody fragment is an antibody which binds to one or more tumor-associated antigens or cell-surface receptors selected from (1)-(89):

(1) BMPR1B;

(2) E16;

(3) STEAP1;

(4) 0772P;

(5) MPF;

(6) Napi3b;

(7) Sema 5b;

(8) PSCA hlg;

(9) ETBR;

(10) MSG783;

(11) STEAP2;

(12) TrpM4;

(13) CRIPTO;

(14) CD21;

(15) CD79b;

(16) FcRH2;

(17) HER2;

(18) NCA;

(19) MDP;

(20) IL20R-alpha;

(21) Brevican;

(22) EphB2R;

(23) ASLG659;

(24) PSCA;

(25) GEDA;

(26) BAFF-R;

(27) CD22;

(28) CD79a;

(29) CXCR5;

(30) HLA-DOB;

(31) P2X5;

(32) CD72;

(33) LY64;

(34) FcRH1;

(35) IRTA2;

(36) TENB2;

(37) PSMA—FOLH1;

(38) SST;

(38.1) SSTR2;

(38.2) SSTR5;

(38.3) SSTR1;

(38.4) SSTR3;

(38.5) SSTR4;

(39) ITGAV;

(40) ITGB6;

(41) CEACAM5;

(42) MET;

(43) MUC1;

(44) CA9;

(45) EGFRvIII;

(46) CD33;

(47) CD19;

(48) IL2RA;

(49) AXL;

(50) CD30—TNFRSF8;

(51) BCMA—TNFRSF17;

(52) CT Ags—CTA;

(53) CD174 (Lewis Y)—FUT3;

(54) CLEC14A;

(55) GRP78—HSPA5;

(56) CD70;

(57) Stem Cell specific antigens;

(58) ASG-5;

(59) ENPP3;

(60) PRR4;

(61) GCC—GUCY2C;

(62) Liv-1—SLC39A6;

(63) 5T4;

(64) CD56—NCMA1;

(65) CanAg;

(66) FOLR1;

(67) GPNMB;

(68) TIM-1—HAVCR1;

(69) RG-1/Prostate tumor target Mindin—Mindin/RG-1;

(70) B7-H4—VTCN1;

(71) PTK7;

(72) CD37;

(73) CD138-SDC1;

(74) CD74;

(75) Claudins—CLs;

(76) EGFR;

(77) Her3;

(78) RON-MST1R;

(79) EPHA2;

(80) CD20-MS4A1;

(81) Tenascin C—TNC;

(82) FAP;

(83) DKK-1;

(84) CD52;

(85) CS1—SLAMF7;

(86) Endoglin—ENG;

(87) Annexin A1—ANXA1;

(88) V-CAM (CD106)-VCAM1;

(89) ASCT2 (SLC1A5).

67. The conjugate according to any one of statements 64 to 66, wherein the antibody or antibody fragment is a cysteine-engineered antibody.

68. The conjugate according to any one of statements 64 to 67, wherein p is an integer from 1 to about 10.

69. The conjugate according to statement 68, wherein p is 1, 2, 3, 4, 5, 6, 7, 8, 9 or 10.

70. A mixture of conjugates according to any one of statements 64 to 69, wherein the average drug loading per antibody in the mixture of antibody-drug conjugates is about 2 to about 20.

71. The conjugate or mixture according to any one of statements 60 to 70, for use in therapy.

72. A pharmaceutical composition comprising the conjugate or mixture of any one of statements 60 to 70 and a pharmaceutically acceptable diluent, carrier or excipient.

73. The conjugate or mixture according to any one of statements 60 to 70, or the pharmaceutical composition according to statement 72, for use in the treatment of a proliferative disease in a subject.

74. The conjugate, mixture or pharmaceutical composition according to statement 73, wherein the disease is cancer.

75. Use of a conjugate or mixture according to any one of statements 60 to 70, or the pharmaceutical composition according to statement 72 in a method of medical treatment.

76. A method of medical treatment comprising administering to a patient the pharmaceutical composition of statement 72.

77. The method of statement 76 wherein the method of medical treatment is for treating cancer.

78. The method of statement 77, wherein the patient is administered a chemotherapeutic agent, in combination with the conjugate.

79. Use of a conjugate or mixture according to any one of statements 60 to 670 in a method of manufacture of a medicament for the treatment of a proliferative disease.

80. A method of treating a mammal having a proliferative disease, comprising administering an effective amount of conjugate or mixture according to any one of statements 60 to 70, or the pharmaceutical composition according to statement 72.

1. A compound with the formula I: 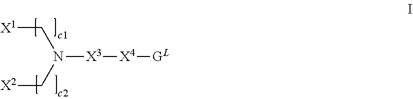
where X¹ and X² are independently selected from a group of formula Ia: 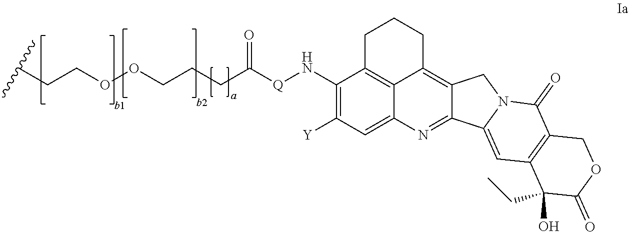
Q is: 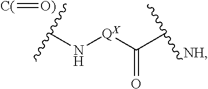
where Q^{X} is such that Q is an amino-acid residue, a dipeptide residue, a tripeptide residue or a tetrapeptide residue;
a=0 to 5, b1=0 to 16, b2=0 to 16, wherein at least b1 or b2=0;
Y is H or F;
c1 is 0 to 5;
c2 is 0 to 5;
X³ is —CH₂— or —C(═O)—;
X⁴ is ^{X3}—(CH₂)_{d1}—(C₂H₄O)ₑ—(CH₂)_{d2}—^{GL}, where d1 is 0 to 5, d2 is 0 to 5 and e is 0 to 16; and
G^{L} is a linker for connecting to a Ligand Unit.

2. The compound according to claim 1, wherein Q is:
(a) an amino acid residue selected from: Phe, Lys, Val, Ala, Cit, Leu, Ile, Arg, and Trp; or
(b) a dipeptide residue selected from:
^{NH}-Phe-Lys-^{C═O},
^{NH}-Val-Ala-^{C═O},
^{NH}-Val-Lys-^{C═O},
^{NH}-Ala-Lys-^{C═O},
^{NH}-Val-Cit-^{C═O},
^{NH}-Phe-Cit-^{C═O},
^{NH}-Leu-Cit-^{C═O},
^{NH}-Ile-Cit-^{C═O},
^{NH}-Phe-Arg-^{C═O},
^{NH}-Trp-Cit-^{C═O}, and
^{NH}-Gly-Val-^{C═O}; or
(c) a tripeptide residue selected from:
^{NH}-Glu-Val-Ala-^{C═O},
^{NH}-Glu-Val-Cit-^{C═O},
^{NH}-αGlu-Val-Ala-^{C═O}, and
^{NH}-αGlu-Val-Cit-^{C═O}; or
(d) a tetrapeptide residue selected from:
^{NH}-Gly-Gly-Phe-Gly ^{C═O}; and
^{NH}-Gly-Phe-Gly-Gly ^{C═O}.

3. The compound according to either claim 1 or claim 2, wherein a is:
(a) 0 to 3; or
(b) 0 or 1; or
(c) 0.

4. The compound according to any one of claims 1 to 3, wherein b1 is:
(a) 0 to 8; or
(b) 0; or
(c) 2; or
(d) 3; or
(e) 4; or
(f) 5; or
(g) 8.

5. The compound according to any one of claims 1 to 3, wherein b2 is:
(a) 0 to 8; or
(b) 0; or
(c) 2; or
(d) 3; or
(e) 4; or
(f) 5; or
(g) 8.

6. The compound according to any one of claims 1 to 5, wherein Y is H.

7. The compound according to any one of claims 1 to 6, wherein X¹ and X² are the same.

8. The compound according to any one of claims 1 to 7, wherein c1 is:
(a) 0 to 3,
(b) 1 or 2; or
(c) 2.

9. The compound according to any one of claims 1 to 8, wherein c2 is:
(a) 0 to 3;
(b) 1 or 2; or
(c) 2.

10. The compound according to any one of claims 1 to 9, wherein c1 and c2 are the same.

11. The compound according to any one of claims 1 to 10, wherein X³ is —C(═O)—.

12. The compound according to any one of claims 1 to 11, wherein d1 is:
(a) 0 to 3;
(b) 1 or 2; or
(c) 2.

13. The compound according to any one of claims 1 to 12, wherein d2 is:
(a) 0 to 3;
(b) 1 or 2; or
(c) 2; or
(d) 0.

14. The compound according to any one of claims 1 to 13, wherein e is:
(a) 0 to 8;
(b) 0;
(c) 2;
(d) 4; or
(e) 8.

15. The compound according to any one of claims 1 to 11, wherein d1+d2 is 2 and e is 0.

16. The compound according to claim 14, wherein each a is 0, each b1 is 0, each b2 is 2, c1 is 2, c2=2, X3=—C(═O)—, d1 is 2, d2 is 0 and e is 0.

17. The compound according to any one of claims 1 to 16, wherein G^{L} is selected from 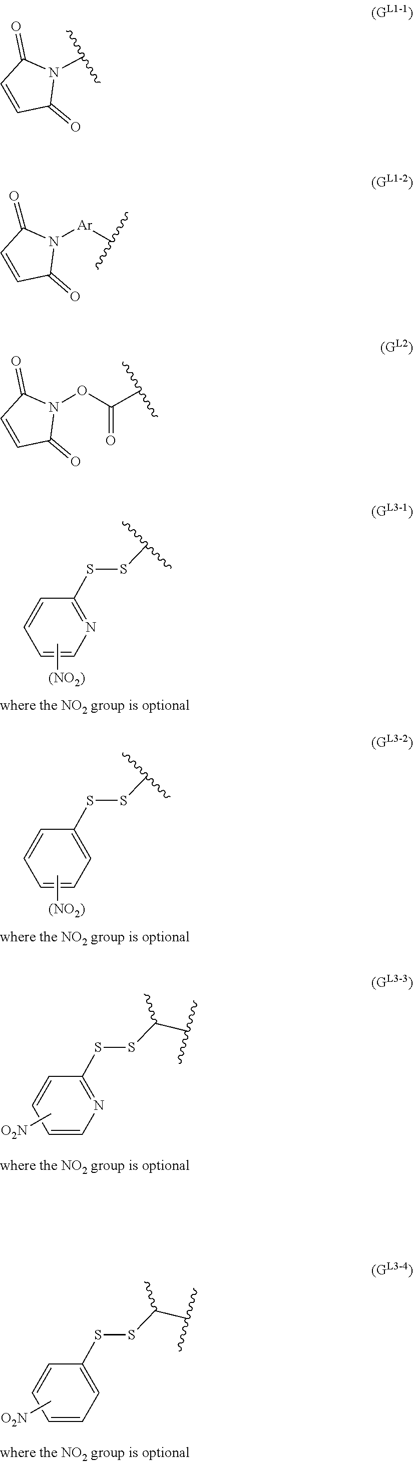 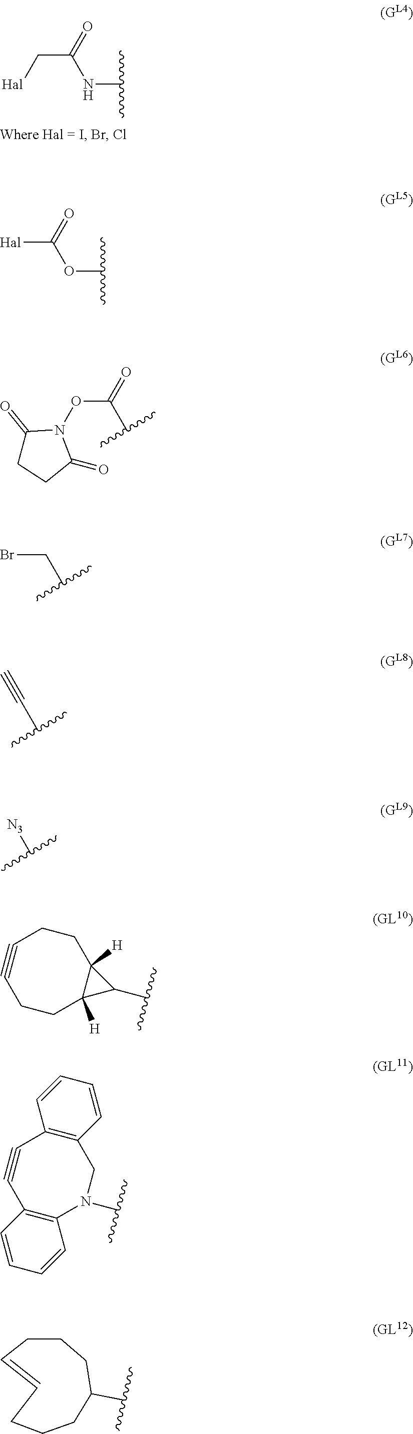 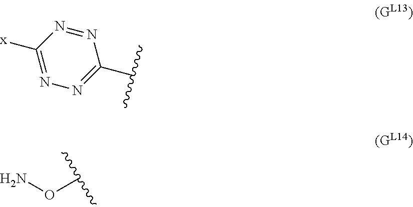
where Ar represents a C₅₋₆ arylene group, and X represents C₁₋₄ alkyl.

18. A compound according to claim 17, wherein G^{L} is selected from G^{L1-1} and G^{L1-2}.

19. A conjugate of formula IV:
L−(D^{L})_{P} (IV)
or a pharmaceutically acceptable salt or solvate thereof, wherein L is a Ligand unit, D^{L} is a Drug Linker unit that is of formula III: 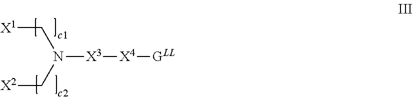
where X¹, X², X³, X⁴, c1 and c2 are as defined in any one of claims 1 to 16;
G^{LL} is a linker connected to a Ligand Unit; and
p is an integer of from 1 to 20.

20. The conjugate according to claim 19, wherein G^{LL} is selected from: 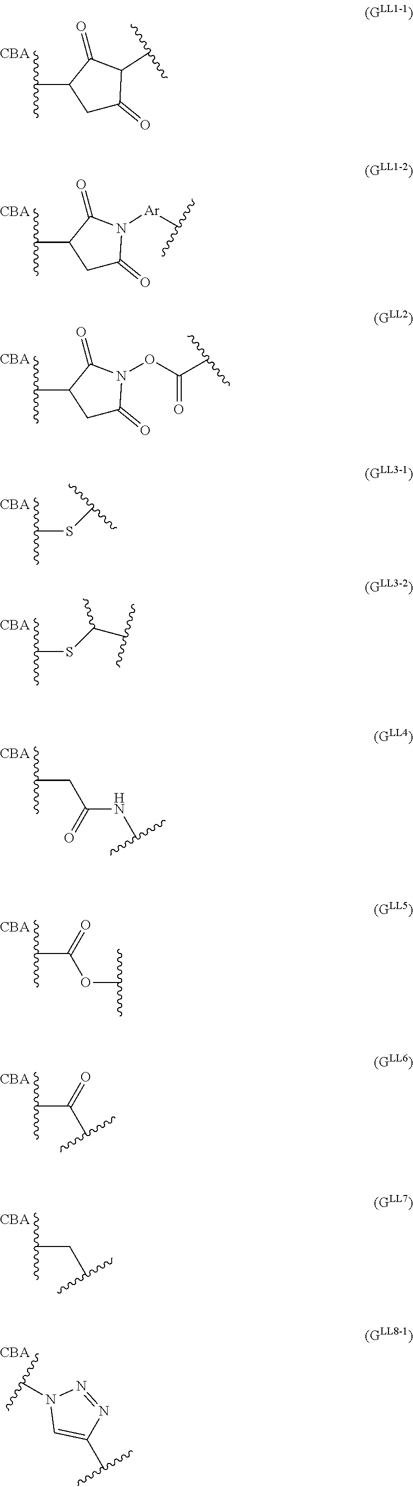 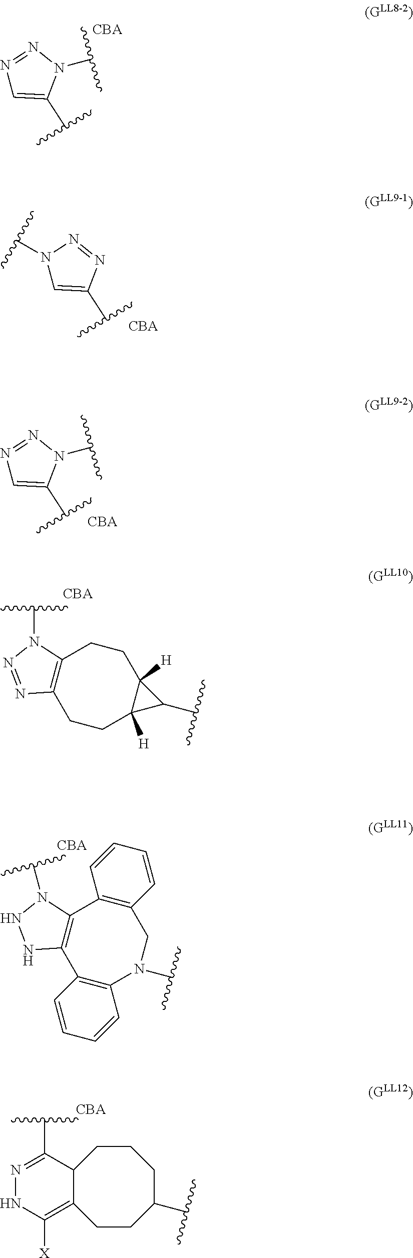 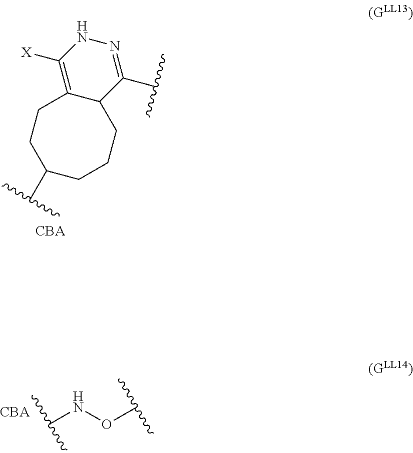
where Ar represents a C₅₋₆ arylene group and X represents C₁₋₄ alkyl.

21. The conjugate according to claim 20, wherein G^{LL} is selected from G^{LL1-1} and G^{LL1-2}.

22. The conjugate according to either claim 19 or 20, wherein the Ligand Unit is an antibody or an active fragment thereof.

23. The conjugate according to claim 22, wherein the p is an integer from 1 to about 10.

24. A mixture of conjugates according to either claim 22 or 23, wherein the average drug loading per antibody in the mixture of antibody-drug conjugates is about 2 to about 20.

25. A pharmaceutical composition comprising the conjugate or mixture of any one of claims 20 to 24 and a pharmaceutically acceptable diluent, carrier or excipient.

26. The conjugate or mixture according to any one of claims 19 to 24, or the pharmaceutical composition according to claim 25, for use in the treatment of a proliferative disease in a subject.

27. The conjugate, mixture or pharmaceutical composition according to claim 26, wherein the disease is cancer.

28. Use of a conjugate or mixture according to any one of claims 19 to 24, or the pharmaceutical composition according to claim 25 in a method of medical treatment.

29. A method of medical treatment comprising administering to a patient the pharmaceutical composition of claim 25.

30. The method of claim 29 wherein the method of medical treatment is for treating cancer.
